(19) United States
(12) Patent Application Publication
(10) Pub. No.: US 2024/0252123 A1
(43) Pub. Date: Aug. 1, 2024

(54) ENVIRONMENTAL FACTORS FOR HEART FAILURE RISK STRATIFICATION

(71) Applicant: Medtronic, Inc., Minneapolis, MN (US)

(72) Inventors: Keara A. Berlin, St. Paul, MN (US); Brandon D. Stoick, Lexington, MN (US); Andrew P. Radtke, Minneapolis, MN (US); Kelvin Mei, Minneapolis, MN (US); Brett D. Jackson, Shoreview, MN (US); Donald R. Musgrove, Minneapolis, MN (US); Tarek D. Haddad, Minneapolis, MN (US); Wade M. Demmer, Coon Rapids, MN (US); Shantanu Sarkar, Roseville, MN (US); Yong K. Cho, Excelsior, MN (US)

(21) Appl. No.: 18/421,457

(22) Filed: Jan. 24, 2024

Related U.S. Application Data
(60) Provisional application No. 63/481,933, filed on Jan. 27, 2023.

(51) Int. Cl. A61B 5/00; A61B 5/0538

(52) U.S. Cl. CPC A61B 5/746; A61B 5/0538; A61B 5/6847; A61B 5/7275; A61B 2560/0252

(57) ABSTRACT

A method uses internal patient data from an implantable medical device (IMD) and environmental factor information associated with cardiovascular diseases as input to a predictive cardiovascular disease software tool for enabling alerts, e.g., generated by a computing system configured to receive data from the IMD. A computing system may receive diagnostic metric data from the IMID and time correlated location data of the patient, e.g., from a smartphone, smartwatch, or other computing device of the user. The computing system may use the patient's location, such as from a user's device such a programmer or patient's computing device, to determine a particulate matter exposure level corresponding to the diagnostic metric data that may then be used as an input to a predictive cardiovascular disease software tool to refine the risk score or risk stratification.

### RELATED APPLICATIONS

This application claims the benefit of U.S. Provisional Application Ser. No. 63/481,933, filed Jan. 27, 2023, the entire contents of each of which are incorporated herein by reference.

### FIELD

The disclosure relates to medical devices, and, more particularly, to medical devices that monitor cardiac health of a patient.

### BACKGROUND

Chronic heart failure (HF) occurs when a heart is unable to consistently pump blood at an adequate rate in response to the filling pressure. To improve the ability of the heart to pump blood, congestive heart failure patients, classified as having New York Heart Association (NYHA) class status of II to IV HF, may require therapy, such as may be provided by implantable medical devices (IMDs) such as implantable cardioverter defibrillators (ICDs), cardiovascular implantable electronic devices (CIEDs), implantable cardiac monitors (ICM), pacemakers, and cardiac resynchronization therapy (CRT) devices that, in some cases, include defibrillation capability (CRT-Ds). Despite using IMDs to improve heart function, some HF patients may require hospitalization. Global health care systems incur billions of dollars each year due to heart failure hospitalizations (HFHs). Identifying patients at risk of HFH to enable timely intervention and prevent expensive hospitalization remains a challenge.

IMDs may be configured to acquire data for a variety of diagnostic metrics that change with HF status and collectively have the potential to signal an increasing risk of HFH. Diagnostic parameter data collected by IMDs may include activity, day and night heart rate (NHR), atrial tachycardia/atrial fibrillation (AT/AF) burden, mean rate during AT/AF, percent CRT pacing, number of shocks, subcutaneous impedance, respiratory rate and effort, premature ventricular contractions (PVC) burden, sleep apnea burden, nighttime posture, chronotropic incompetence and intrathoracic impedance. Additionally, preset or programmable thresholds for diagnostic metrics, when crossed, trigger a notification, referred to as device observation. Each device observation is recorded in an IMD report.

### SUMMARY

In general, this disclosure describes techniques that include use of both internal patient data from an IMD and environmental factor information associated with cardiovascular diseases as input to a predictive cardiovascular disease software tool for enabling alerts, e.g., generated by a computing system configured to receive data from the IMD. For example, particulate matter data may be used as input to a predictive cardiovascular disease software tool. For example, wildfires are increasing in frequency and intensity due to climate change and will continue to become more frequent and intense as climate change progresses. Increased wildfires are causing increasing levels of human exposure to the particulate matter contained in wildfire smoke, which research has shown is associated with an increased incidence of various heart problems. The computing system may receive diagnostic metric data from the IMD and time-correlated location data of the patient, e.g., from a smartphone, smartwatch, or other computing device of the user. The computing system may use the patient's location, such as from a user's device such a programmer or patient's computing device, to determine a particulate matter exposure level corresponding to the diagnostic metric data that may then be used as an input to a predictive cardiovascular disease software tool to refine the risk score or risk stratification.

In general, three main categories related to HF are physiologic information related to compensatory mechanisms, signs and symptoms, and triggering conditions. Fluid levels (from impedance), heart rate, HRV, respiratory rate can be considered as physiologic. Activity reduction, nighttime posture changes, and respiratory rate are related to signs and symptoms. Intensity of activity, Occurrence of AF with rapid ventricular rate, non-compliance, infection in lungs, etc. may all be considered as triggers. Environmental factors may be considered as additional triggers. For example, an increase in pollutants may lead to trouble in O2 exchange in lungs and can lead increase in Oxygen demand and then a set of compensatory mechanism activation in HF patients which may lead to a decompensation event.

Environmental factor information as an input to the predictive cardiovascular disease software tool may potentially improve the accuracy of the model by providing it with additional information that it can use to make more accurate predictions. This can lead to more accurate diagnoses and treatment recommendations, which can improve patient outcomes. A model with environmental factor information may be able to make more specific diagnoses, as it has more information to work with. This can be particularly useful in cases where there are multiple possible diagnoses that are difficult to distinguish based on a limited set of input features. In some cases, the system may make environmental based recommendations, such as to stay indoors. A model with environmental factor information input may be more interpretable, as it has more features to indicate why a particular diagnosis or treatment recommendation was made. This can help physicians understand the reasoning behind the model's predictions and make more informed clinical decisions. A model with environmental factor information input may also reduce reliance on subjective measures: Environmental factor information input is objective and quantifiable and may help reduce the reliance on subjective measures (e.g., physician judgment) in the diagnosis process, which can reduce variability and improve the reproducibility of the diagnosis process. In general, the environmental factor information may give machine learning model in the predictive cardiovascular disease software tool more information to work with which can result in more accurate predictions and thus generalize better to real world data.

When the system predicts a worsening HF event, a first acute goal is to prevent the HF event from getting worse. In the acute phase, such prevention includes providing therapy to manage fluid. Outside of the acute phase, the system may evaluate contributing factors and provide therapeutic suggestions to deal with that. So, if environmental factor is one of the trigger reasons as identified by the model, one can provide recommendations on how to deal with the environmental factor or other contributory factors.

In one example, the disclosure describes a system comprising: one or more input/output devices; and one or more processors configured to: receive, from an implantable medical device via the one or more input/output devices, a plurality of diagnostic parameters of a patient associated with heart failure, wherein at least one of the plurality of diagnostic parameters is measured by the implantable medical device; obtain environmental factor information associated with heart failure for the patient via the one or more input/output devices; determine a heart failure (HF) risk score indicating probability of occurrence of a heart failure event of the patient, wherein the HF risk score is derived using a model that uses the plurality of diagnostic parameters monitored over time and the environmental factor information; and in response to the HF risk score exceeding a threshold value, provide an alert or a suggestion to modify a therapy delivered to the patient by the implantable medical device via the one or more input/output devices.

In another example, the disclosure describes a method for determining a heart failure (HF) risk score of a patient indicating a probability of occurrence of a heart failure event, the method comprising: receiving a plurality of diagnostic parameters of a patient associated with heart failure from an implantable device, wherein at least one of the plurality of diagnostic parameters is measured by and retrieved from the implantable medical device; obtaining environmental factor information associated with heart failure for the patient; determining a heart failure (HF) risk score indicating probability of occurrence of a heart failure event, wherein the HF risk score is derived using a model that uses the plurality of diagnostic parameters monitored over time and the environmental factor information; and in response to the HF risk score exceeding a threshold value, providing an alert or a suggestion to modify a therapy delivered to the patient by the implantable medical device.

In another example, the disclosure describes a system comprising: an implantable medical device comprising one or more sensors configured to monitor over time at least one primary diagnostic parameter associated with heart failure; and an external device interacting with the implantable medical device to obtain the at least one primary diagnostic parameter associated with heart failure; and a server comprising: a processor configured to receive the at least one primary diagnostic parameter from the implantable medical device; obtain environmental factor information associated with heart failure for the patient; determine a heart failure (HF) risk score indicating probability of occurrence of a heart failure event of the patient, wherein the HF risk score is derived using a model that uses the at least one primary diagnostic parameter monitored over time and the environmental factor information; and in response to the HF risk score exceeding a threshold value, provide an alert or a suggestion to modify a therapy delivered to the patient by the implantable medical device.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual drawing illustrating an example system configured to transmit diagnostic information indicative of heart failure, the system including an implantable medical device (IMD) coupled to implantable medical leads.

FIG. 2A is a conceptual drawing illustrating the example IMD and leads of FIG. 1 in conjunction with a heart.

FIG. 2B is a conceptual drawing illustrating the example IMD of FIG. 1 coupled to a different configuration of implantable medical leads in conjunction with a heart.

FIG. 3 is a functional block diagram illustrating an example configuration of the IMD of FIG. 1.

FIG. 4 is a functional block diagram illustrating an example configuration of an external device that facilitates user communication with the IMD.

FIG. 5 is a block diagram illustrating an example system that includes a device, such as a server, and one or more computing devices that are coupled to the IMD and external device shown in FIG. 1 via a network.

FIG. 6 illustrates an example user interface that includes a heart failure risk status that indicates a risk of the patient for hospitalization due to heart failure.

FIG. 7 illustrates an example method for patient risk stratification using internal device data and at least one environmental factor.

FIG. 8 is a flow diagram of example techniques for generating a heart failure risk score using a Bayesian Belief Network model.

FIG. 9 is a flowchart that illustrates an example method for patient risk stratification using internal device data and at least one environmental factor.

### DETAILED DESCRIPTION

This disclosure describes techniques for acquiring and utilizing diagnostic information, such as patient metrics, and at least one environmental factor by an external monitoring device, such as a server like a cloud-based service, from a remote monitoring device, such as an implantable medical device (IMD), to differentiate between heart failure risk levels. Congestive heart failure may occur gradually over time due to heart disease, patient inactivity, cardiac arrhythmias, hypertension, and other conditions. Often, however, a relatively rapid worsening of the patient's condition, e.g., a decompensation, precipitates hospitalization and, in some cases, death.

The external monitoring device, such as a server, may differentiate between heart failure risk levels using at least one environmental factor, as well as diagnostic information, such as patient metrics, from an IMD. Environmental factors may influence heart failure risk levels. For example, particulate mater has been found to increase heart failure risk levels. Particulate matter, or PM, is a type of air pollution made up of tiny particles suspended in the air. These particles can come from a variety of sources, including car exhaust, industrial emissions, and wildfires. Wildfires, for example, are increasing in frequency and intensity due to climate change and will continue to become more frequent and more intense as climate change progresses. Increased wildfires are causing increasing levels of human exposure to particulate matter contained in wildfire smoke, which research has shown is associated with increased incidence of a variety of heart problems.

Particulate matter may be inhaled into the lungs and can cause a variety of health problems, including increased risk of heart attack. The risk of heart attack increases with prolonged exposure to high levels of particulate matter, particularly in people who are already at risk for heart disease due to factors such as high blood pressure, diabetes, or a family history of heart disease. Particulate matter may trigger a HF worsening cascade due to the potential increase in O2 demand. Other forms of air pollution, such as high ozone levels, also impact the cardiovascular system and may increase heart failure risk levels.

Another environmental factor that has been found to increase heart failure risk levels is external temperature. Studies have found that the risk of HF increases during periods of extreme cold or heat. Large changes in temperature may trigger compensatory mechanisms in the human body. Healthy people may be able to easily handle such temperature swings, however HF patients are closer to a HF “cliff” so a large swing in in a compensatory mechanism may lead to a decompensation. More broadly seasonal variations are observed and that may be dependent on the geographical location. Coping with winter in Minnesota is very different in physiological mechanisms compared to coping with winter in Florida. Similarly, coping with summer in Florida is different in physiological mechanisms, such as sweating, compared to coping with summer in Minnesota. To manage these environmental risks, risk stratification systems may be modified to include such environmental factors as additional factors in calculating risk stratification. Such environmental factor information, such as particulate matter levels, may be obtained using by location information, such as zip code, address, or latitude and longitude coordinates. Location information may be obtained from an external device, such as a programmer or other device, or from a patient computing device, such as a smart phone. Location information may be obtained from a database that stores a patient's address. The determined location information may be used to obtain environmental factor information. Such environmental factor information may be obtained from a service using an application programming interface (API) that provides access to the environmental factor information. For example, particulate matter levels may be obtained from a public source of particulate matter levels, such as AirNow.gov or waqi.info, and temperature levels may be obtained from a public source of temperature data, such as climate.gov.

Environmental factor information, such as particulate matter levels, may be also obtained using sensors near the patient. Examples of particulate matter sensors include laser-based sensors, optical sensors, and electrical mobility sensors. Examples of temperature sensors include thermistors and infrared (IR) sensors.

One or more implantable medical devices (IMDs) or external medical devices (described hereinafter with respect to a single IMD, although any example herein may be performed by a system including any number of implantable or external medical devices), e.g., a pacemaker, cardioverter and/or defibrillator, or a monitoring device that does not provide therapy, such as the Reveal LINQ™ insertable cardiac monitor, commercially available from Medtronic plc, of Dublin, Ireland, may automatically generate and store patient data regarding patient metrics. The patient metrics may include, as examples, therapy use statistics (e.g., pacing or shocks), thoracic impedance, heart rate, heart rate variability, patient activity, and a percentage of time receiving cardiac resynchronization therapy. Other example patient metrics include weight, blood pressure, respiration rate, sleep apnea burden (which may be derived from respiration rate or heart rate changes), temperature, ischemia burden, the occurrence, frequency or duration cardiac events, and sensed cardiac intervals (e.g., heart rate or Q-T intervals). Examples of cardiac events may include atrial and ventricular tachyarrhythmias. Another example patient metric is the atrial tachycardia/fibrillation (AT/AF) burden and ventricular rate during AT/AF. The concentration or levels of various substances, such as blood glucose, hematocrit, troponin and/or brain natriuretic peptide (BNP) levels, within the patient may also be used as one or more patient metrics.

The IMD may provide diagnostic information to one or more users via one or more devices, such as IMD programmers or other computing devices. The diagnostic information may be related to, generated from, or may even include the one or more patient metrics. The diagnostic information may include, as examples, values of the patient metrics and raw data used to derive the values of the patient metrics.

An external monitoring device, such as a server or other computing device, acquires and utilizes the patient metrics associated with diagnostic information from the IMD, and uses the patient metrics, along with environmental factor information, to generate heart failure risk scores and differentiate between heart failure risk status alerts. The scheme of alerts described herein applies to all types of risk segmentations. Heart failure risk statuses may be segmented by drawing cut-offs on numeric scores. Default cut-offs for Low, Medium, High statuses may be customized for population level data. However, these cut-offs can additionally or alternatively be customized by a user for performance at an individual patient level. Furthermore, if necessary, more than three levels can be created (e.g., Very High and/or Very Low as an additionally level) by the user and cut-off for each can be customized. Along the same lines, two (or more) levels (e.g., Low and Medium) can be merged to create a two-level system.

In some examples, the external monitoring device may receive a transmission of patient metric data for a number of periods, e.g., days, from the IMD, and may generate a periodic heart failure risk status (HFRS) for the periods based on the diagnostic data for the period. The external monitoring device may receive also obtain environmental factor information such as from a public database using geographic information. The external monitoring device may also differentiate the HFRS based on the maximum periodic HFRS within a lookback window prior to the current period. For example, the external monitoring device may differentiate the alerts as being either high alerts, high ongoing alerts, medium alerts and medium ongoing alerts. In some examples, the external monitoring device may differentiate HFRS as being a high HFRS, a high new HFRS, a medium HFRS and a medium new HFRS.

Such additional HFRS differentiation of the present disclosure may assist in prioritization of patients, determining patient conditions, assessing the effectiveness of a current therapy, and determining whether therapy adjustments may be necessary. For example, during monitoring of patients, knowing whether an event is an ongoing event, not an ongoing event, a new event, or not a new event enables more efficient prioritization and treatment of patients by the clinician.

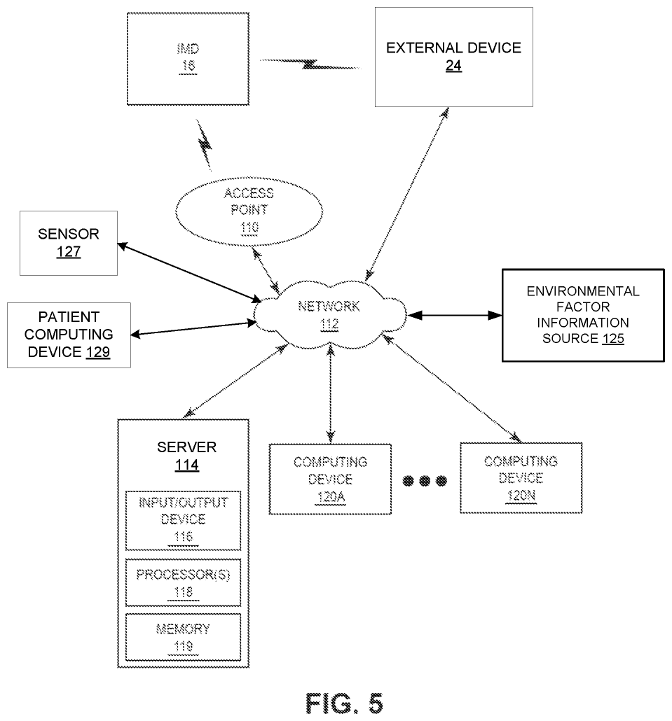

In some examples, a HFRS notification, e.g., a new high, may be sent to a user according to their preferences, e.g., via email, pager, or text message (such as Short Message Service (SMS) or multimedia messages (MMS)). Moreover, processing circuitry of an IMD system may select a type of notification based on a determined HFRS differentiation, e.g., new versus ongoing, in some examples according to the techniques of this disclosure. The notification may indicate the HFRS, and in some cases the differentiation. For example, the processing circuitry may select a notification pushed to a personal device of a user, e.g., a text message or email, for HFRS that are new and/or more severe, such as a new high HFRS, while other less new and/or severe HFRS notifications are collected and accessible at the user's convenience via a web service, e.g., the system of FIG. 5. The user may select a type of notification based on the HFRS differentiation, e.g., provide user preferences. For example, the user may only want to receive text messages for a new high and receive emails for all other alerts. In another example, the user may want to receive a message on their pager for all high new HFRS and medium new HFRS alerts and wait to access all other alerts via the web service.

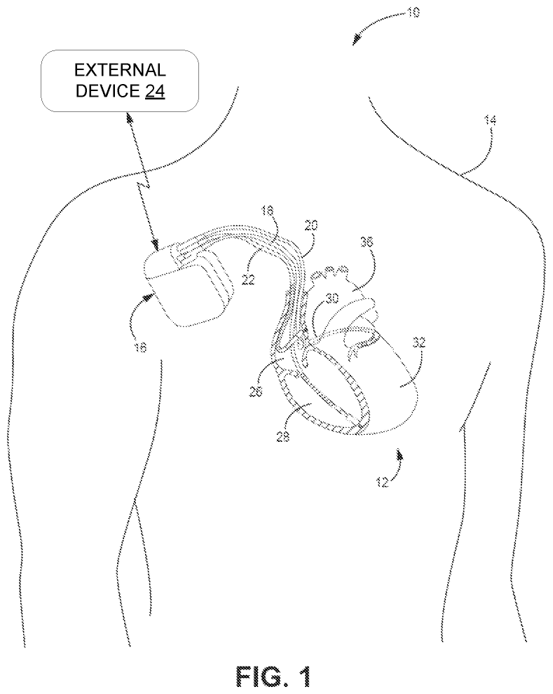

FIG. 1 is a conceptual drawing illustrating an example system 10 configured to transmit diagnostic information indicative of heart failure of patient 14. In the example of FIG. 1, system 10 includes IMD 16, which is coupled to leads 18, 20, and 22, and external device 24, such as a programmer. IMD 16 may be, for example, an implantable pacemaker, cardioverter, and/or defibrillator that provides electrical signals to heart 12 via electrodes coupled to one or more of leads 18, 20, and 22. Patient 14 is ordinarily, but not necessarily a human patient.

In the example of FIG. 1, leads 18, 20, 22 extend into the heart 12 of patient 14 to sense electrical activity of heart 12 and/or deliver electrical stimulation to heart 12. Leads 18, 20, and 22 may also be used to detect a thoracic impedance indicative of fluid volume in patient 14, respiration rates, sleep apnea, or other patient metrics. Respiration metrics, e.g., respiration rates, tidal volume, and sleep apnea, may also be detectable via an electrogram, e.g., based on a signal component in a cardiac electrogram that is associated with respiration. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

In some examples, system 10 may additionally or alternatively include one or more leads or lead segments (not shown in FIG. 1) that deploy one or more electrodes within the vena cava, or other veins. Furthermore, in some examples, system 10 may additionally or alternatively include temporary or permanent epicardial or subcutaneous leads with electrodes implanted outside of heart 12, instead of or in addition to transvenous, intracardiac leads 18, 20 and 22. Such leads may be used for one or more of cardiac sensing, pacing, or cardioversion/defibrillation. For example, these electrodes may allow alternative electrical sensing configurations that provide improved or supplemental sensing in some patients. In other examples, these other leads may be used to detect intrathoracic impedance as a patient metric for identifying a heart failure risk or fluid retention levels.

IMD 16 may sense electrical signals attendant to the depolarization and repolarization of heart 12 via electrodes (not shown in FIG. 1) coupled to at least one of the leads 18, 20, 22. In some examples, IMD 16 provides pacing pulses to heart 12 based on the electrical signals sensed within heart 12. The configurations of electrodes used by IMD 16 for sensing and pacing may be unipolar or bipolar. IMD 16 may detect arrhythmia of heart 12, such as tachycardia or fibrillation of the atria 26 and 36 and/or ventricles 28 and 32, and may also provide defibrillation therapy and/or cardioversion therapy via electrodes located on at least one of the leads 18, 20, 22. In some examples, IMD 16 may be programmed to deliver a progression of therapies, e.g., pulses with increasing energy levels, until a fibrillation of heart 12 is stopped. IMD 16 may detect fibrillation employing one or more fibrillation detection techniques known in the art.

In addition, IMD 16 may monitor the electrical signals of heart 12 for patient metrics stored in IMD 16 and/or used in generating the heart failure risk level. IMD 16 may utilize two of any electrodes carried on leads 18, 20, 22 to generate electrograms of cardiac activity. In some examples, IMD 16 may also use a housing electrode of IMD 16 (not shown) to generate electrograms and monitor cardiac activity. Although these electrograms may be used to monitor heart 12 for potential arrhythmias and other disorders for therapy, the electrograms may also be used to monitor the condition of heart 12. For example, IMD 16 may monitor heart rate (nighttime and daytime), heart rate variability, ventricular or atrial intrinsic pacing rates, indicators of blood flow, or other indicators of the ability of heart 12 to pump blood or the progression of heart failure.

In some examples, IMD 16 may also use any two electrodes of leads 18, 20, and 22 or the housing electrode to sense the intrathoracic impedance of patient 14. As the tissues within the thoracic cavity of patient 14 increase in fluid content, the impedance between two electrodes may also change. For example, the impedance between an RV coil electrode and the housing electrode may be used to monitor changing intrathoracic impedance.

IMD 16 may use this impedance to create a fluid index. As the fluid index increases, more fluid may be more likely to be retained within patient 14 and heart 12 may be stressed to keep up with moving the greater amount of fluid. Therefore, this fluid index may be a patient metric transmitted in higher resolution diagnostic data or used to generate the heart failure risk level. By monitoring the fluid index in addition to other patient metrics, IMD 16 may be able to reduce the number of false positive heart failure identifications relative to what might occur when monitoring only one or two patient metrics. Furthermore, IMD 16, along with other networked computing devices described herein, may facilitate remote monitoring of patient 14, e.g., monitoring by a health care professional when the patient is not located in a healthcare facility or clinic associated with the health care professional, during a post-hospitalization period. An example system for measuring thoracic impedance and determining a fluid index is described in U.S. Patent Publication No. 2010/0030292 to Sarkar et al., entitled, “DETECTING WORSENING HEART FAILURE BASED ON IMPEDANCE MEASUREMENTS,” which published on Feb. 4, 2010, and is incorporated herein by reference in its entirety.

IMD 16 may also communicate with external device 24. In some examples, external device 24 may be a handheld computing device, computer workstation, or networked computing device. External device 24 may include a user interface that receives input from a user. In other examples, the user may also interact with external device 24 remotely via a networked computing device. The user may interact with external device 24 to communicate with IMD 16. For example, the user may interact with external device 24 to send an interrogation request and retrieve patient metrics or other diagnostic information from IMD 16. A user may also interact with external device 24 to program IMD 16, e.g., select values for operational parameters of IMD 16. Although the user is a physician, technician, surgeon, electrophysiologist, or other healthcare professional, the user may be patient 14 in some examples.

For example, the user may use external device 24 to retrieve information from IMD 16 regarding patient metric data and/or the heart failure risk level. Although external device 24 may retrieve this information after submitting an interrogation request, IMD 16 may push or transmit the heart failure risk level, for example, if the heart failure risk level indicates a change in patient treatment is necessary. For example, the risk level may be determined based on a predetermined number of patient metrics exceeding their representative thresholds or a weighted score for each of the patient metrics for exceeding one or more thresholds. Additionally, or alternatively, the risk level may be determined by a model such as a Bayesian Belief Network, or other probability technique, or other artificial intelligence (AI) model such as a convolutional neural network (CNN) or long short-term memory (LSTM) model using the values or stratified states of each automatically detected patient metric.

One method for determining heart failure risk status is described in U.S. Publication No. 2012/0253207 A1, entitled “Heart Failure Monitoring,” by Sarkar et al., which is incorporated herein by reference in its entirety. In this example, the “High” risk level is associated with 15% or greater risk that the patient will be hospitalized. The “Low” category may in this example represent diagnostic evaluations where the metric state for all the patient metrics was mostly “Low” (e.g., a risk score of less than ˜5%). The “Medium” category comprises of all other metric state combinations that did not get classified as either “High” or “Low.”

Although IMD 16 may generate the heart failure risk level, IMD 16 may transmit the patient metric data and external device 24 or another computing device or system that receives the parameter data from IMD 16, e.g., via external device 24, may generate the heart failure risk level in other examples. External device 24 may present an alert to the user with the higher or lower resolution diagnostic information, e.g., the heart failure risk level and/or other patient metric data. The patient metric data may include intracardiac or intravascular pressure, activity, posture, respiration, or thoracic impedance.

As another example, the user may use external device 24 to retrieve information from IMD 16 regarding the performance or integrity of IMD 16 or other components of system 10, such as leads 18, 20 and 22, or a power source of IMD 16. In some examples, any of this information may be presented to the user as an alert (e.g., a notification or instruction). Further, alerts may be pushed from IMD 16 to facilitate alert delivery whenever external device 24 is detectable by IMD 16. IMD 16 may wirelessly transmit alerts, or other higher or lower resolution diagnostic information, to facilitate immediate notification of the heart failure condition.

External device 24 may also allow the user to define how IMD 16 senses, detects, and manages each of the patient metrics. For example, the user may define the frequency of sampling or the evaluation window used to monitor the patient metrics. In addition, the user may use external device 24 to set each metric threshold used to monitor the status of each patient metric. The metric thresholds may be used to determine when each of the patient metrics has reached a magnitude indicative of being at risk for heart failure. In some examples, when a patient metric exceeds its respective metric threshold, the metric may be counted in the predetermined number used to create the HFRS, which may be presented as a risk level. For example, if two of the eight patient metrics exceed their thresholds, the heart failure risk level may be described as a high risk level for patient 14 to be hospitalized within thirty days. This heart failure risk level may indicate that patient 14 is at an increased risk of heart failure if the predetermined number of patient metrics exceeding their respective thresholds is one or more. The hospitalization monitoring period may be adjusted to be longer or shorter than thirty days, e.g., fourteen days or forty days.

The risk level may be a predetermined number that is set to different values for patients of differing age, weight, cardiac condition, or any number of other risk factors. In other examples, the predetermined number may be set to a different number or a risk level percentage (fraction). In this manner, the predetermined number may represent a preset fraction of unweighted or weighted metrics exceeding a threshold with respect to the total number of monitored metrics. External device 24 may be used to set this predetermined number or any other factors used to generate and interpret the heart failure risk level.

In other examples, the risk level may be determined by the sum, average, or other combination of weighted scores for each of the patient metrics. Each patient metric may have one or more metric-specific thresholds that stratify the state of the metric. Since some states or metrics may be more indicative of the risk of hospitalization, these states and/or metrics may provide a greater contribution to the determined risk level. For example, a high risk state for intrathoracic impedance may have a weighted score that is double that of a high risk state for patient inactivity. In other words, intrathoracic impedance may be a greater risk factor to the patient than patient inactivity. In some examples, a probability analysis may be performed on some or all of the patient metrics to determine the probability that patient 14 will be hospitalized for heart failure. For example, a Bayesian Belief Network may be applied to the values of the patient metrics and environmental factor information to determine the risk level, e.g., the probability, that patient 14 will be admitted to the hospital for heart failure.

In some examples, one or more patient metrics may be collected or detected outside of IMD 16. Patient metrics collected outside of IMD 16 may be referred to as “non-device metrics.” These non-device metrics may be useful for some patients in determining the heart failure risk level before hospitalization, during hospitalization, and/or during the post-hospitalization period. These non-device metrics may be collected, e.g., received via patient input or electronic transmission from another device, and may be analyzed similar to any other patient metrics described herein. Example non-device metrics may include patient weight, medication compliance, consumed food, liquid intake, activity durations, pain levels, pain locations, urinary or fecal voiding events, or any other non-device metrics that may describe or otherwise characterize the health of patient 14.

IMD 16 and external device 24 may communicate via wireless communication using any techniques known in the art. Examples of communication techniques may include, for example, radiofrequency (RF) telemetry, but other communication techniques such as magnetic coupling are also contemplated. In some examples, external device 24 may include a programming head that may be placed proximate to the body of the patient near the IMD 16 implant site in order to improve the quality or security of communication between IMD 16 and external device 24.

As described herein, IMD 16 automatically detects a plurality of patient metrics from patient 14. These patient metrics may alone, or in combination, be indicative of heart failure of patient 14. The patient metrics may include, as examples, thoracic impedance, heart rate variability, the number, frequency or duration of atrial fibrillation after cardioversion therapy, ventricular rate during persistent atrial fibrillation, night heart rate, or any other metrics detectable from patient 14 or based on the treatment of patient 14.

As one example, the heart failure risk level may indicate a high risk of hospitalization when a predetermined number of the plurality of automatically detected patient metrics, such as two or more automatically detected patient metrics, each exceed their respective metric-specific threshold. As another example, the heart failure risk level may indicate a medium risk of hospitalization when a predetermined number of the plurality of automatically detected patient metrics, such as only one automatically detected patient metric, exceeds its respective metric-specific threshold. In an additional example, the heart failure risk level may indicate a low risk of hospitalization when none of the plurality of automatically detected patient metrics exceeds their respective metric-specific thresholds.

IMD 16 may automatically detect each of the patient metrics and store them within the IMD for later transmission. Although IMD 16 may automatically detect eight different patient metrics in some examples, IMD 16 may detect more or less patient metrics in other examples. For example, the patient metrics may include two or more of a thoracic fluid index, an atrial fibrillation duration, a ventricular contraction rate during atrial fibrillation, a patient activity, a nighttime heart rate, a heart rate variability, a cardiac resynchronization therapy (CRT) percentage (e.g., the percentage of cardiac cycles for which cardiac resynchronization pacing was provided), or the occurrence of or number of therapeutic electrical shocks. The metric-specific thresholds may include at least two of a thoracic fluid index threshold of approximately 60, an atrial fibrillation duration threshold of approximately 6 hours, a ventricular contraction rate threshold approximately equal to 90 beats per minute for 24 hours, a patient activity threshold approximately equal to 1 hour per day for seven consecutive days, a nighttime heart rate threshold of approximately 85 beats per minute for seven consecutive days, a heart rate variability threshold of approximately 40 milliseconds for seven consecutive days, a cardiac resynchronization therapy percentage threshold of 90 percent for five of seven consecutive days, or an electrical shock threshold of 1 electrical shock. In other examples, each of the metric-specific thresholds may have various different values determined by the clinician, based on previous data from other patients, or determined based on a healthy state of patient 14. In some examples, the metric-specific thresholds may be rate of change thresholds or relative change thresholds, e.g., a heart rate variability that is decreasing faster than a predetermined rate, or a predetermined amount or percentage less than a recently identified variability value.

In some examples, the heart failure risk level may be determined with probability models that determine the probability of hospitalization based on the values of all patient metrics. In this manner, each of the patient metric values may contribute to the risk level regardless of whether a metric-specific threshold is exceeded. For example, IMD 16 may generate a heart failure risk level with a Bayesian Belief Network based on the plurality of automatically generated patient metrics. The risk level may include general levels, e.g., a high risk, medium risk, or low risk of hospitalization, or numerical indications, e.g., a percent probability that patient 14 will be hospitalized.

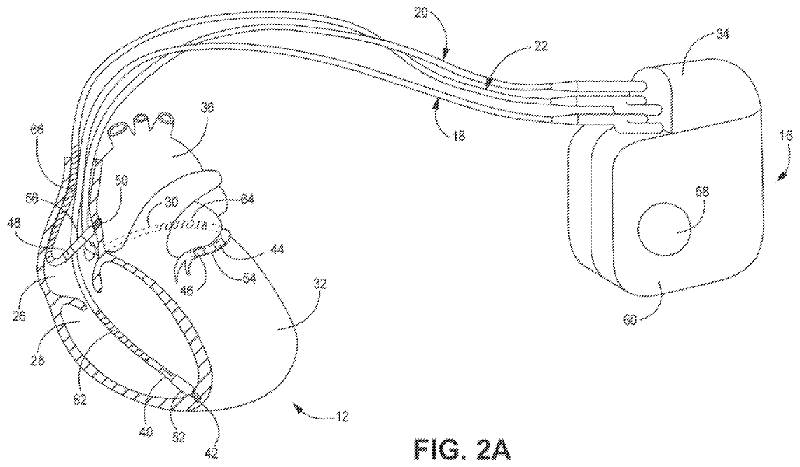

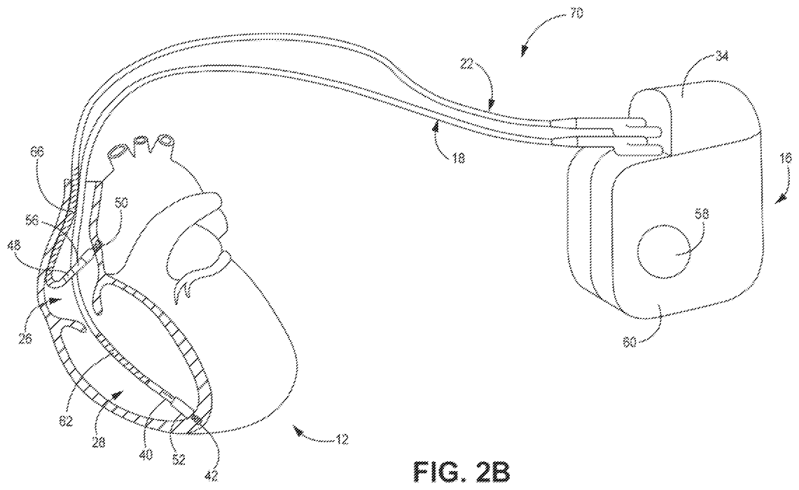

FIG. 2A is a conceptual drawing illustrating IMD 16 and leads 18, 20, and 22 of system 10 in greater detail. As shown in FIG. 2A, IMD 16 is coupled to leads 18, 20, and 22. Leads 18, 20, 22 may be electrically coupled to a signal generator, e.g., stimulation generator, and a sensing module of IMD 16 via connector block 34. In some examples, proximal ends of leads 18, 20, 22 may include electrical contacts that electrically couple to respective electrical contacts within connector block 34 of IMD 16. In addition, in some examples, leads 18, 20, 22 may be mechanically coupled to connector block 34 with the aid of set screws, connection pins, snap connectors, or another suitable mechanical coupling mechanism.

Each of the leads 18, 20, 22 includes an elongated insulative lead body, which may carry a number of concentric coiled conductors separated from one another by tubular insulative sheaths. Bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. In the illustrated example, there are no electrodes located in left atrium 36. However, other examples may include electrodes in left atrium 36.

Electrodes 40, 44, and 48 may take the form of ring electrodes, and electrodes 42, 46 and 50 may take the form of extendable helix tip electrodes mounted retractably within insulative electrode heads 52, 54 and 56, respectively. In other examples, one or more of electrodes 42, 46 and 50 may take the form of small circular electrodes at the tip of a tined lead or other fixation element. Leads 18, 20, 22 also include elongated electrodes 62, 64, 66, respectively, which may take the form of a coil. Each of the electrodes 40, 42, 44, 46, 48, 50, 62, 64 and 66 may be electrically coupled to a respective one of the coiled conductors within the lead body of its associated lead 18, 20, 22, and thereby coupled to respective ones of the electrical contacts on the proximal end of leads 18, 20 and 22.

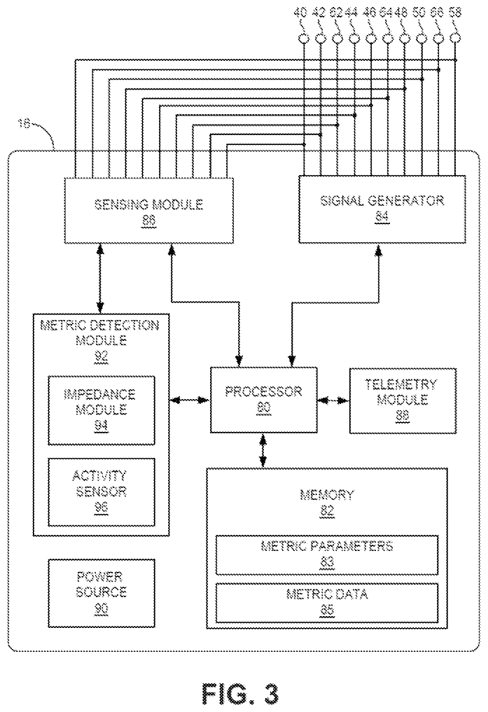

In some examples, as illustrated in FIG. 2A, IMD 16 includes one or more housing electrodes, such as housing electrode 58, which may be formed integrally with an outer surface of hermetically-sealed housing 60 of IMD 16, or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. As described in further detail with reference to FIG. 3, housing 60 may enclose a signal generator that generates therapeutic stimulation, such as cardiac pacing pulses and defibrillation shocks, as well as a sensing module for monitoring the rhythm of heart 12.

IMD 16 may sense electrical signals attendant to the depolarization and repolarization of heart 12 via electrodes 40, 42, 44, 46, 48, 50, 62, 64 and 66. The electrical signals are conducted to IMD 16 from the electrodes via the respective leads 18, 20, 22. IMD 16 may sense such electrical signals via any bipolar combination of electrodes 40, 42, 44, 46, 48, 50, 62, 64 and 66. Furthermore, any of the electrodes 40, 42, 44, 46, 48, 50, 62, 64 and 66 may be used for unipolar sensing in combination with housing electrode 58. The combination of electrodes used for sensing may be referred to as a sensing configuration or electrode vector.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes. The combination of electrodes used for delivery of stimulation or sensing, their associated conductors and connectors, and any tissue or fluid between the electrodes, may define an electrical path.

The configuration of system 10 illustrated in FIGS. 1 and 2A is merely one example. In other examples, a system may include epicardial leads and/or subcutaneous electrodes instead of or in addition to the transvenous leads 18, 20, 22 illustrated in FIG. 1. Further, IMD 16 need not be implanted within patient 14. In examples in which IMD 16 is not implanted in patient 14, IMD 16 may sense electrical signals and/or deliver defibrillation pulses and other therapies to heart 12 via percutaneous leads that extend through the skin of patient 14 to a variety of positions within or outside of heart 12. Further, external electrodes or other sensors may be used by IMD 16 to deliver therapy to patient 14 and/or sense and detect patient metrics used to generate the high and lower resolution diagnostic information, e.g., a heart failure risk level.

In addition, in other examples, a system may include any suitable number of leads coupled to IMD 16, and each of the leads may extend to any location within or proximate to heart 12. For example, systems in accordance with this disclosure may include three transvenous leads located as illustrated in FIGS. 1 and 2, and an additional lead located within or proximate to left atrium 36. As another example, systems may include a single lead that extends from IMD 16 into right atrium 26 or right ventricle 28, or two leads that extend into a respective one of the right ventricle 26 and right atrium 26. An example of a two lead type of system is shown in FIG. 2B. Any electrodes located on these additional leads may be used in sensing and/or stimulation configurations.

In some examples, a system may additionally or alternatively include one more subcutaneous pacing and/or defibrillation devices coupled to extravascular leads, leadless pacing devices configured for implantation within a heart chamber, and/or implantable or external monitoring devices that monitor patient parameters but do not provide therapy, such as the above-mentioned Reveal LINQ™ insertable cardiac monitor.

Any of electrodes 40, 42, 44, 46, 48, 50, 62, 64, 66, and 58 may be utilized by IMD 16 to sense or detect patient metrics used to generate the heart failure risk level for patient 14. Typically, IMD 16 may detect and collect patient metrics from those electrode vectors used to treat patient 14. For example, IMD 16 may derive an atrial fibrillation duration, heart rate, and heart rate variability metrics from electrograms generated using vectors that are also used to deliver pacing therapy. However, IMD 16 may utilize other electrodes to detect these types of metrics from patient 14 when other electrical signals may be more appropriate for therapy.

In addition to electrograms of cardiac signals, any of electrodes 40, 42, 44, 46, 48, 50, 62, 64, 66, and 58 may be used to sense non-cardiac signals. For example, two or more electrodes may be used to measure an impedance within the thoracic cavity of patient 14. This intrathoracic impedance may be used to generate a fluid index patient metric that indicates the amount of fluid building up within patient 14. Since a greater amount of fluid may indicate increased pumping loads on heart 12, the fluid index may be used as an indicator of heart failure risk. IMD 16 may periodically measure the intrathoracic impedance to identify a trend in the fluid index over days, weeks, months, and even years of patient monitoring.

In general, the two electrodes used to measure the intrathoracic impedance may be located at two different positions within the chest of patient 14. For example, coil electrode 62 and housing electrode 58 may be used as the sensing vector for intrathoracic impedance because electrode 62 is located within RV 28 and housing electrode 58 is located at the IMD 16 implant site generally in the upper chest region. However, other electrodes spanning multiple organs or tissues of patient 14 may also be used, e.g., an additional implanted electrode used only for measuring thoracic impedance.

FIG. 2B is a conceptual diagram illustrating another example system 70, which is similar to system 10 of FIGS. 1 and 2, but includes two leads 18, 22, rather than three leads. Leads 18, 22 are implanted within right ventricle 28 and right atrium 26, respectively. System 70 shown in FIG. 2B may be useful for physiological sensing and/or providing pacing, cardioversion, or other therapies to heart 12. Detection of patient metrics and differentiation of heart failure risk status according to this disclosure may be performed in two lead systems in the manner described herein with respect to three lead systems. In other examples, a system similar to systems 10 and 70 may only include one lead (e.g., any of leads 18, 20 or 22) to deliver therapy and/or sense and detect patient metrics related to monitoring risk of heart failure. Alternatively, diagnostic data may be implemented in systems utilizing subcutaneous leads, subcutaneous IMDs, or external medical devices. Although FIGS. 1-2 provide an exemplary IMD 16 implantation in which the notification differentiation described below may be implemented, it is understood that IMD 16 and its associated electrodes may be implemented in other locations of the patient's body, and may include leads or may be leadless.

FIG. 3 is a functional block diagram illustrating an example configuration of IMD 16. In the illustrated example, IMD 16 includes a processor 80, memory 82, metric detection module 92, signal generator 84, sensing module 86, telemetry module 88, and power source 90. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 80 may include processing circuitry including any one or more of a microprocessor, a controller, a digital signal processor (DSP), an application specific integrated circuit (ASIC), a field-programmable gate array (FPGA), or equivalent discrete or analog logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof, e.g., may be embodied as software or firmware executed on processing circuitry.

Processor 80 controls signal generator 84 to deliver therapy to heart 12 according to a therapy parameters and programs which may be stored in memory 82. Signal generator 84 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. In the illustrated example, signal generator 84 is configured to generate and deliver electrical stimulation therapy to heart 12. For example, signal generator 84 may deliver defibrillation shocks to heart 12 via at least two electrodes 58, 62, 64, 66. Signal generator 84 may deliver pacing pulses via ring electrodes 40, 44, 48 coupled to leads 18, 20, and 22, respectively, and/or helical electrodes 42, 46, and 50 of leads 18, 20, and 22, respectively. In some examples, signal generator 84 delivers pacing, cardioversion, or defibrillation stimulation in the form of electrical pulses. In other examples, signal generator may deliver one or more of these types of stimulation in the form of other signals, such as sine waves, square waves, or other substantially continuous time signals.

Signal generator 84 includes circuitry, such as charge pumps, capacitors, current mirrors, or other signal generation circuitry for generating a pulse or other signal. Signal generator 84 may include a switch module and processor 80 may use the switch module to select, e.g., via a data/address bus, which of the available electrodes are used to deliver defibrillation pulses or pacing pulses. The switch module may include a switch array, switch matrix, multiplexer, or any other type of switching device suitable to selectively couple stimulation energy to selected electrodes.

Electrical sensing module 86 monitors signals from at least one of electrodes 40, 42, 44, 46, 48, 50, 58, 62, 64 or 66 in order to monitor electrical activity of heart 12, impedance, or another electrical phenomenon. Sensing may be done to determine heart rates or heart rate variability, or to detect arrhythmias or other electrical signals. Sensing module 86 may include one or more filters, amplifiers, analog-to-digital converters, or other sensing circuitry.

Sensing module 86 may also include a switch module to select which of the available electrodes are used to sense the heart activity, depending upon which electrode combination, or electrode vector, is used in the current sensing configuration. In some examples, processor 80 may select the electrodes that function as sense electrodes, i.e., select the sensing configuration, via the switch module within sensing module 86. Sensing module 86 may include one or more detection channels, each of which may be coupled to a selected electrode configuration for detection of cardiac signals via that electrode configuration. Some detection channels may be configured to detect cardiac events, such as P- or R-waves, and provide indications of the occurrences of such events to processor 80.

Processor 80 may implement programmable counters. If IMD 16 is configured to generate and deliver pacing pulses to heart 12, such counters may control the basic time intervals associated with DDD, VVI, DVI, VDD, AAI, DDI, DDDR, VVIR, DVIR, VDDR, AAIR, DDIR, CRT, and other modes of pacing.

Intervals defined by processor 80 may include atrial and ventricular pacing escape intervals, refractory periods during which sensed P-waves and R-waves are ineffective to restart timing of the escape intervals, and the pulse widths of the pacing pulses. As another example, processor 80 may withhold sensing from one or more channels of sensing module 86 for a time interval during and after delivery of electrical stimulation to heart 12. The durations of these intervals may be determined by processor 80 in response to stored data in memory 82. Processor 80 may also determine the amplitude of the cardiac pacing pulses.

Interval counters implemented by processor 80 may be reset upon sensing of R-waves and P-waves with detection channels of sensing module 86. In examples in which IMD 16 provides pacing, signal generator 84 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. In such examples, processor 80 may reset the interval counters upon the generation of pacing pulses by signal generator 84, and thereby control the basic timing of cardiac pacing functions, including anti-tachyarrhythmia pacing.

The value of the count present in the interval counters when reset by sensed R-waves and P-waves may be used by processor 80 to measure the durations of R-R intervals, P-P intervals, P-R intervals and R-P intervals, which are measurements that may be stored in memory 82. Processor 80 may use the count in the interval counters to detect a tachyarrhythmia event, such as atrial fibrillation (AF), atrial tachycardia (AT), ventricular fibrillation (VF), or ventricular tachycardia (VT). These intervals may also be used to detect the overall heart rate, ventricular contraction rate, and heart rate variability. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia.

In some examples, processor 80 may determine that tachyarrhythmia has occurred by identification of shortened R-R (or P-P) interval lengths. Generally, processor 80 detects tachycardia when the interval length falls below 220 milliseconds (ms) and fibrillation when the interval length falls below 180 ms. These interval lengths are merely examples, and a user may define the interval lengths as desired, which may then be stored within memory 82. This interval length may need to be detected for a certain number of consecutive cycles, for a certain percentage of cycles within a running window, or a running average for a certain number of cardiac cycles, as examples.

In the event that processor 80 detects an atrial or ventricular tachyarrhythmia based on signals from sensing module 86, and an anti-tachyarrhythmia pacing regimen is desired, timing intervals for controlling the generation of anti-tachyarrhythmia pacing therapies by signal generator 84 may be used by processor 80 to control the operation of the escape interval counters therein and to define refractory periods during which detection of R-waves and P-waves is ineffective to restart the escape interval counters for the an anti-tachyarrhythmia pacing. In the event that processor 80 detects an atrial or ventricular tachyarrhythmia based on signals from sensing module 86, and a cardioversion or defibrillation shock is desired, processor 80 may control the amplitude, form and timing of the shock delivered by signal generator 84.

Memory 82 may be configured to store a variety of operational parameters, therapy parameters, sensed and detected data, and any other information related to the therapy and treatment of patient 14. In the example of FIG. 3, memory 82 also includes metric parameters 83 and metric data 85. Metric parameters 83 may include all of the parameters and instructions required by processor 80 and metric detection module 92 to sense and detect each of the patient metrics used to generate the diagnostic information transmitted by IMD 16. Metric data 85 may store all of the data generated from the sensing and detecting of each patient metric. In this manner, memory 82 stores a plurality of automatically detected patient metrics as the data required to generate a risk level of patient 14 being admitted to the hospital due to heart failure. Metric detection module 92 may include processing circuitry that is different than, or at least partially overlapping with, the processing circuitry of processor 80, and may be embodied as software or firmware executed by such processing circuitry. In some examples, metric detection module 92 may be a software/firmware module executed by processor 80.

Metric parameters 83 may include definitions of each of the patient metrics automatically sensed or measured by metric detection module 92. These definitions may include instructions regarding what electrodes or sensors to use in the detection of each metric, the sample rate, calibration schemes, metric-specific thresholds, and any other related information. In one example, the patient metrics for which metric parameters are stored as metric parameters 83 may include a thoracic fluid index (or a thoracic impedance), an atrial tachycardia or fibrillation burden, a ventricular contraction rate during atrial fibrillation, a patient activity, a nighttime heart rate, a difference between night and day heart rate, a heart rate variability, a cardiac resynchronization therapy percentage, a bradyarrhythmia pacing therapy percentage (in a ventricle and/or atrium), and number or frequency of electrical shock events. In other examples, other patient metrics may be stored that may be useful in the detection of heart failure risk, e.g., blood pressure, right ventricular pressure, pulmonary artery pressure, patient temperature, lung volume, lung tidal volume, lung density, breathing rate or even biomarkers such as a brain natriuretic peptide (BNP), troponin, or related surrogates. In such examples, IMD 16 may include or be coupled to sensors known in the art for detecting such metrics. In some examples, the atrial tachycardia or fibrillation burden may be a time of the event, a percent or amount of time over a certain period, a number of episodes, or even a frequency of episodes.

Metric parameters 83 may also store a metric-specific threshold for each of the patient metrics automatically detected by metric detection module 92. Metric thresholds may be predetermined and held constant over the entire monitoring of patient 14. In some examples, however, metric thresholds may be modified by a user during therapy or processor 80 may modify one or more metric thresholds to compensate for certain patient conditions. For example, a heart rate threshold may be changed over the course of monitoring if the normal or baseline heart rate has changed during therapy. Processor 80 may also suggest or present metric parameter 83 or other therapy or sensing settings or programming changes. In one example, if metric detection module 92 identifies a patient with new atrial fibrillation, processor may recommend programming changes intended to respond to atrial fibrillation, such as programming anti-tachycardia pacing (ATP) or pacing modes that promote hemodynamically beneficial pacing, e.g., cardiac resynchronization therapy (CRT), despite rate variability during atrial fibrillation. Example recommendations include “Turn on Reactive ATP Settings” or “Turn on Effective CRT during AT/AF.” Effectiveness in CRT is described in U.S. Pat. No. 8,750,998 to Ghosh et al., entitled, “EFFECTIVE CAPTURE TEST,” which issued on Jun. 10, 2014, and is incorporated herein by reference in its entirety.

In one example, these metric-specific thresholds may include a thoracic fluid index threshold of approximately 60, an atrial fibrillation burden threshold of approximately 6 consecutive hours, a ventricular contraction rate threshold approximately equal to 90 beats per minute for 24 hours, a patient activity threshold approximately equal to 1 hour per day for seven consecutive days, a nighttime heart rate threshold of approximately 85 beats per minute for seven consecutive days, a heart rate variability threshold of approximately 40 milliseconds for seven consecutive days, a cardiac resynchronization therapy percentage threshold of 90 percent for five of seven consecutive days, and an electrical shock number threshold of 1 electrical shock. These thresholds may be different in other examples, and may be configured by a user, e.g., a clinician, for an individual patient.

Processor 80 may alter the method with which patient metrics are stored in memory 82 as metric data 85. In other words, processor 80 may store the automatically detected patient metrics with a dynamic data storage rate. The dynamic storage rate may be higher when processor 80 needs to transmit higher resolution diagnostic information and lower when processor 80 needs to transmit lower resolution diagnostic information. For example, processor 80 may store patient metrics in memory 82 every minute or hour when processor 80 is transmitting higher resolution diagnostic information. However, processor 80 may only store patient metrics in memory 82 once a day, for example, when processor 80 is only transmitting lower resolution diagnostic information that does not necessitate more frequent data. In addition to the dynamic storage rate, the rate at which metric detection module 92 automatically detects each patient metric may be altered to match the dynamic storage rate. In this manner, metric detection module 92 may not waste energy detecting patient metrics if the higher frequency data would just be discarded.

Metric detection module 92 may, for example, transmit lower resolution diagnostic information (e.g., a heart failure risk level) that is based on the patient metrics and whether any of the metrics exceed the respective specific metric thresholds. Any time that an automatically detected patient metric exceeds their respective metric threshold, the patient metric is counted in the risk level. In one example, if two or more of the eight patient metrics exceed their respective metric threshold, then the risk level would be classified as a high risk. In other examples, the risk level may include a numerical value such as 2 out of 8 (e.g., threshold exceeding metrics out of the total number of monitored metrics). The higher the risk level, the more likely that patient 14 is at risk to be admitted to the hospital within a predefined time period, e.g., admitted to the hospital within a predefined number of days. For example, each threshold exceeding metric counted in the predetermined number may contribute to a higher risk level of heart failure. In this example, a risk level of 1 out of 8 may indicate a medium risk of hospitalization, a risk level of 2 out of 8 may indicate a high risk of hospitalization, and a risk level of 3 out of 8 may indicate a very high risk of hospitalization.

It is also noted that exceeding a metric threshold does not require that the detected value of the patient metric becomes greater than the magnitude of the threshold. For some patient metrics, exceeding the metric threshold may occur when the value of the patient metric drops below the metric threshold. Therefore, each threshold may be a boundary that triggers the metric's inclusion in the heart failure risk level any time that the metric threshold is crossed. In other examples, as described above, the risk level may be calculated as a sum of weighted metrics such that some metrics may impact the risk level greater than other metrics (e.g., a trans-thoracic impedance may be weighted double that of other metrics). This use of thresholds for determining the risk levels may be considered heuristic logic.

In this manner, metric detection module 92 may automatically detect each of the patient metrics and store them within metric data 85 for later transmission. Metric detection module 92 may be any type of hardware (e.g., a specialized circuit or processor) or a software module executed by a processor (e.g., processor 80). Metric parameters 83 may generally store one metric-specific threshold per patient metric, but other examples may include several thresholds to apply depending on other patient conditions, delivered therapies, or even the importance of one patient metric. For example, the thoracic fluid index determined from sensed intrathoracic impedance may be subject to two separate metric thresholds each counting towards the predetermined number of the heart failure risk level. The first thoracic fluid index threshold may be set to a value of 60, but the second thoracic fluid index threshold may be set to a value of 100. If the thoracic fluid index metric exceeds the first thoracic fluid index threshold of 60, the fluid index metric may be counted in the heart failure risk level. If the fluid index also crosses the second thoracic fluid index threshold of 100, the fluid index metric may be counted in the heart failure risk level a second time. In this manner, the heart failure risk level may weight more extreme values of some metrics more heavily than other metrics. In one example, the fluid index value may be a unitless number using a recent intrathoracic impedance, a short term mean impedance, an impedance variability value, and a duration value. Example fluid index values and impedance measurements are described in U.S. Patent Application No. 2010/0030292. As the intrathoracic impedance remains low, the fluid index may increase. Conversely, as the intrathoracic impedance remains high, the fluid index may decrease. In this manner, the fluid index value maybe a numerical representation of retained fluid that is specific to patient 14. In other examples, the intrathoracic impedance may be alternatively used.

In some examples, a statistical or probability analysis may be performed on some or all of the patient metrics to determine the probability that patient 14 will be hospitalized for heart failure. In this manner, the heart failure risk level may be determined without utilizing thresholds for each of the detected patient metrics. Instead, metric detection module 92 or processor 80 may examine the values of each of the patient metrics for relative contributions to the possibility that patient 14 is at a higher risk of being hospitalized. For example, a Bayesian Belief Network may use the values of the patient metrics to determine the risk level that patient 14 will be admitted to the hospital for heart failure. Such statistical analysis is described in PCT Patent Publication No. WO 2011/126823A1 entitled, “METHOD AND APPARATUS FOR MONITORING TISSUE FLUID CONTENT FOR USE IN AN IMPLANTABLE CARDIAC DEVICE,” which is incorporated by reference herein in its entirety.

Metric data 85 is a portion of memory 82 that may store some or all of the patient metric data that is sensed and detected by metric detection module 92. Metric data 85 may store the data for each metric on a rolling basis during an evaluation window. The evaluation window may only retain recent data and delete older data from the evaluation window when new data enters the evaluation window. In this manner, the evaluation window may include only recent data for a predetermined period of time. Processor 80 may access metric data when necessary to retrieve and transmit patient metric data and/or generate heart failure risk levels. In addition, metric data 85 may store heart failure risk levels or other generated information related to the heart failure risk of patient 14. Although metric parameters 83 and/or metric data 85 may consist of separate physical memories, these components may simply be an allocated portion of the greater memory 82.

Metric detection module 92 may automatically sense and detect each of the patient metrics. Metric detection module 92 may then generate diagnostic information, e.g., risk levels, based on the patient metrics. For example, metric detection module 92 may measure the thoracic impedance, analyze an electrogram of heart 12, monitor the electrical stimulation therapy delivered to patient 14, or sense the patient activity. It is noted that functions attributed to metric detection module 92 herein may be embodied as software, firmware, hardware or any combination thereof. In some examples, metric detection module 92 may at least partially be a software process executed by processor 80. Metric detection module 92 may sense or detect any of the patient metrics used as a basis for generating the heart failure risk level or otherwise indication of heart failure score or status or that patient 14 is at risk for hospitalization. In one example, metric detection module 92 may compare each of the patient metrics to their respective metric-specific thresholds defined in metric parameters 83 to generate the heart failure risk level. Metric detection module 92 may automatically detect two or more patient metrics. In other examples, metric detection module 92 may detect eight different patient metrics.

In one example, metric detection module 92 may analyze electrograms received from sensing module 86 to detect an atrial fibrillation or atrial tachycardia, and determine atrial tachycardia or fibrillation burden, e.g., duration, as well as a ventricular contraction rate during atrial fibrillation. Metric detection module 92 may also analyze electrograms in conjunction with a real-time clock, patient posture or activity signal, e.g., from activity sensor 96, and/or other physiological signals indicative of when a patient is asleep or awake to determine a nighttime (or sleeping) heart rate or a daytime (or awake) heart rate or a difference between the day and night heart rate, and also analyze electrograms to determine a heart rate variability, or any other detectable cardiac events from one or more electrograms. As described above, metric detection module 92 may use peak detection, interval detection, or other methods to analyze the electrograms.

In addition, metric detection module 92 may include and/or control impedance module 94 and activity sensor 96. Impedance module 94 may be used to detect the thoracic impedance used to generate the thoracic fluid index. As described herein, impedance module 94 may utilize any of the electrodes of FIG. 1, 2 or 3 to take intrathoracic impedance measurements. In other examples, impedance module 94 may utilize separate electrodes coupled to IMD 16 or in wireless communication with telemetry module 88. Once impedance module 94 measures the intrathoracic impedance of patient 14, metric detection module 92 may generate the thoracic fluid index and compare the index to the thoracic fluid index threshold defined in metric parameters 83.

Activity sensor 96 may include one or more accelerometers or other devices capable of detecting motion and/or position of patient 14. Activity sensor 96 may therefore detect activities of patient 14 or postures engaged by patient 14. Metric detection module 92 may, for example, monitor the patient activity metric based on the magnitude or duration of each activity and compare the determined metric data to the activity threshold defined in metric parameters 83. The activity patient metric may then be used to generate the heart failure risk level.

In addition to detecting events of patient 14, metric detection module 92 may also detect certain therapies delivered by signal generator 84, e.g., as directed by processor 80. Metric detection module 92 may monitor signals through signal generator 84 or receive therapy information directly from processor 80 for the detection. Example patient metrics detected by this method may include a cardiac resynchronization therapy percentage or metrics related to delivery of electrical shocks.

The cardiac resynchronization therapy (CRT) metric may be the amount or percentage of time each day, or an amount of percentage of cardiac cycles, as examples, that IMD 16 delivers cardiac resynchronization therapy to heart 12. Low CRT amounts or percentages may indicate that beneficial therapy is not being delivered and that adjustment of therapy parameters, e.g., an atrioventricular delay or a lower pacing rate, may improve therapy efficacy. In one example, higher CRT amounts or percentages may indicate that heart 12 is sufficiently pumping blood through the vasculature with the aid of therapy to prevent fluid buildup. In examples of other types of cardiac pacing (non-CRT) or stimulation therapy, higher therapy percentages may indicate that heart 12 is unable to keep up with blood flow requirements.

An electrical shock may be a defibrillation event or other high energy shock used to return heart 12 to a normal rhythm. The metric related electrical shocks may be a number or frequency of electrical shocks, e.g., a number of shocks within a period of time. Metric detection module 92 may detect these patient metrics as well and compare them to a cardiac resynchronization therapy percentage and shock event threshold, respectively, defined in metric parameters 83 to determine when each patient metric has become critical. In one example, the electrical shock event metric may become critical when a threshold number of shocks is delivered, e.g., within a time period, or even when patient 14 even receives one therapeutic shock.

Metric detection module 92 may include additional sub-modules or sub-routines that detect and monitor other patient metrics used to monitor patient 14 and/or generate the heart failure risk level. In some examples, metric detection module 92, or portions thereof, may be incorporated into processor 80 or sensing module 86. In other examples, raw data used to produce patient metric data may be stored in metric data 85 for later processing or transmission to a device. A device may then produce each patient metric from the raw data, e.g., electrogram or intrathoracic impedance. In other examples, metric detection module 92 may additionally receive data from one or more implanted or external devices used to detect each metric which IMD 16 may store as metric data.

In some examples, the patient metric thresholds used to generate the risk levels may change over time, e.g., the patient metric thresholds may either be modified by a user or automatically changed based on other patient conditions. Telemetry module 88 may receive commands from external device 24, for example, to modify one or more metric parameters 83 (e.g., metric creation instructions or metric-specific thresholds). In some examples, processor 80 may adjust a metric-specific threshold if certain conditions are present in patient 14. For example, the threshold may be adjusted if patient 14 is experiencing certain arrhythmias or data contained in cardiac electrograms change, e.g., there is a deviation in ST elevations or presence of pre-ventricular contractions, in such a manner that requires a change in the threshold.

Processor 80 may generate the heart failure risk level based upon the patient metrics sensed, detected, and stored in metric data 85 of memory 82. For example, processor 80 may continually update the heart failure risk level as metric detection module 92 updates each patient metric. In other examples, processor 80 may periodically update the heart failure risk level according to an updating schedule. Processor 80 may compare each of the automatically detected patient metrics to their respective metric-specific thresholds and automatically generate the heart failure risk level based on the comparison. The comparison of metrics to thresholds and the like can be performed on the external device 24, the patient computing device 129, or the server 114.

Processor 80 may also compare the heart failure risk level to the predetermined number stored in memory 82. The predetermined number may indicate when patient 14 is at an increased risk of heart failure. The predetermined number may be a percentage or a number of patient metrics exceeding the respective metric threshold. At this stage, the risk level may be considered critical. Although a clinician may be presented with the heart failure risk level at any time, processor 80 may push the heart failure risk level to a clinician or other healthcare professional in an alert. This immediacy may be necessary because a critical risk level indicates that heart failure may be imminent in a large number of patients with the same patient metric levels. Therefore, a clinician may receive the transmitted diagnostic information of the critical risk level and initiate alternative treatment to prevent patient 14 from hospitalization.

In some examples, external device 24, a computing device, e.g., a smart phone or other computing device of patient 14, or a server may additionally or alternatively include a metric detection module similar to metric detection module 92 described herein. For example, external device 24 may generate the risk level based on diagnostic information, including patient metric values, transmitted by IMD 16. However, processor 80 may still collect and store the data for each patient metric or even organize and format the patient metric data before transmitting the patient metrics in metric data 85 to the device. In addition, processor 80 may transmit the metric thresholds with the patient metric data so that any device may generate heart failure risk levels specific to patient 14.

As described above, processor 80 may provide an alert to a user, e.g., of external device 24, regarding the data from any patient metric and/or the heart failure risk level. In one example, processor 80 may provide an alert with the heart failure risk level when external device 24 or another device communicates with IMD 16. This communication may be in the form of an interrogation request that is sent to IMD 16. In response to the interrogation request, processor 80 may transmit higher resolution diagnostic information if patient 14 is hospitalized or lower resolution diagnostic information if patient 14 is in the post-hospitalization period. In other examples, processor 80 may push an alert to external device 24 or another device whenever the heart failure risk level becomes critical via transmission by telemetry module 88. Alternatively, IMD 16 may directly indicate to patient 14 that medical treatment is needed due to a critical heart failure risk level. IMD 16 may include a speaker to emit an audible sound through the skin of patient 14 or a vibration module that vibrates to notify patient 14 of needed medical attention. Processor 80 may choose this action, for example, if the alert cannot be sent because of no available connection.

Telemetry module 88 includes any suitable hardware, firmware, software or any combination thereof for communicating with another device, such as external device 24 (FIG. 1). Under the control of processor 80, telemetry module 88 may receive downlink telemetry from and send uplink telemetry to external device 24 with the aid of an antenna, which may be internal and/or external. Processor 80 may provide the data (higher or lower resolution diagnostic information) to be uplinked to external device 24 and the control signals for the telemetry circuit within telemetry module 88, e.g., via an address/data bus. In some examples, telemetry module 88 may provide received data to processor 80 via a multiplexer.

In some examples, processor 80 may transmit atrial and ventricular heart signals, e.g., EGMs, produced by atrial and ventricular sense amplifier circuits within sensing module 86 to external device 24. External device 24 may interrogate IMD 16 to receive the heart signals. Processor 80 may store heart signals within memory 82, and retrieve stored heart signals from memory 82. Processor 80 may also generate and store marker codes indicative of different cardiac events that sensing module 86 detects, and transmit the marker codes to external device 24.

In some examples, IMD 16 may signal external device 24 to further communicate with and pass the alert through a network such as the Medtronic CareLink® Network developed by Medtronic, Inc., of Minneapolis, MN, or some other network linking patient 14 to a clinician. In this manner, a computing device or user interface of the network may be the external computing device that delivers the alert, e.g., patient metric data in the form of higher resolution diagnostic information or heart failure risk level in the form of lower resolution diagnostic information, to the user. IMD 16 may spontaneously transmit the diagnostic information to the network or in response to an interrogation request from a user. In other examples, one or more steps in the generation of the heart failure risk level may occur within a device external of patient 14, e.g., within external device 24 or a server networked to external device 24. In this manner, IMD 16 may detect and store patient metrics before transmitting the patient metrics to a different computing device. Patient metrics may, in some examples, be compared to means, medians, or outliers from other patients to determine when the patient is at a high risk of hospitalization for heart failure. If one of the automatically detected patient metrics exceeds its respective metric-specific threshold, processor 80 may control telemetry module to transmit that patient metric and possibly other patient metrics to allow the clinician to more accurately diagnose the problem with patient 14.

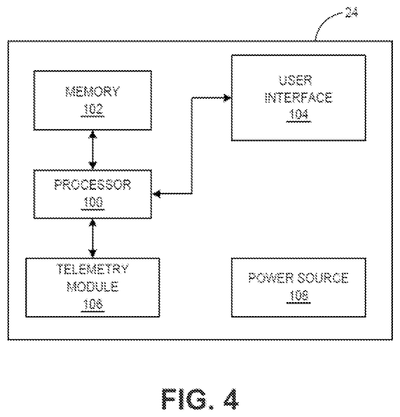

FIG. 4 is a functional block diagram illustrating an example configuration of external device 24. As shown in FIG. 4, external device 24 may include a processor 100, memory 102, user interface 104, telemetry module 106, and power source 108. External device 24 may be a dedicated hardware device with dedicated software for programming of IMD 16. Alternatively, external device 24 may be an off-the-shelf computing device running an application that enables external device 24 to program IMD 16.

A user may use external device 24 to configure the operational parameters of and retrieve data from IMD 16 (FIG. 1). The clinician may interact with external device 24 via user interface 104, which may include display to present graphical user interface to a user, and a keypad or another mechanism for receiving input from a user. In addition, the user may receive an alert or notification from IMD 16 indicating the heart failure risk level and/or patient metrics via external device 24. In other words, external device 24 may receive higher or lower resolution diagnostic information from IMD 16.

Processor 100 can take the form one or more microprocessors, DSPs, ASICs, FPGAS, programmable logic circuitry, or the like, and the functions attributed to processor 100 herein may be embodied as hardware, firmware, software or any combination thereof. Memory 102 may store instructions that cause processor 100 to provide the functionality ascribed to external device 24 herein, and information used by processor 100 to provide the functionality ascribed to external device 24 herein. Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. A removable memory may also allow patient data to be easily transferred to another computing device, or to be removed before external device 24 is used to program therapy for another patient.

External device 24 may communicate wirelessly with IMD 16, such as using RF communication or proximal inductive interaction. This wireless communication is possible through the use of telemetry module 106, which may be coupled to an internal antenna or an external antenna. An external antenna that is coupled to external device 24 may correspond to the programming head that may be placed over heart 12, as described above with reference to FIG. 1. Telemetry module 106 may be similar to telemetry module 88 of IMD 16 (FIG. 4).

Telemetry module 106 may also be configured to communicate with another computing device via wireless communication techniques, or direct communication through a wired connection. Examples of local wireless communication techniques that may be employed to facilitate communication between external device 24 and another computing device include RF communication according to the 802.11 or Bluetooth specification sets, infrared communication, e.g., according to the IrDA standard, or other standard or proprietary telemetry protocols. In this manner, other devices may be capable of communicating with external device 24 without needing to establish a secure wireless connection. An additional computing device in communication with external device 24 may be a networked device such as a server capable of processing information retrieved from IMD 16.

In this manner, telemetry module 106 may transmit an interrogation request to telemetry module 88 of IMD 16. Accordingly, telemetry module 106 may automatically receive diagnostic information, may receive diagnostic information selected by the request, or may receive diagnostic information based on already entered patient status to IMD 16. The diagnostic information may include patient metric values or other detailed information from telemetry module 88 of IMD 16. The diagnostic information may include an alert or notification of the heart failure risk level from telemetry module 88 of IMD 16. The alert may be automatically transmitted, or pushed, by IMD 16 when the heart failure risk level becomes critical. In addition, the alert may be a notification to a healthcare professional, e.g., a clinician or nurse, of the risk level and/or an instruction to patient 14 to seek medical treatment for the potential heart failure condition that may require hospitalization if left untreated. In response to receiving the alert, user interface 104 may present the alert to the healthcare professional regarding the risk level or present an instruction to patient 14 to seek medical treatment.

Either in response to pushed heart failure information, e.g., the risk level or patient metrics, or requested heart failure information, user interface 104 may present the patient metrics and/or the heart failure risk level to the user. In some examples, user interface 104 may also highlight each of the patient metrics that have exceeded the respective one of the plurality of metric-specific thresholds. In this manner, the user may quickly review those patient metrics that have contributed to the identified heart failure risk level.

Upon receiving the alert via user interface 104, the user may also interact with user interface 104 to cancel the alert, forward the alert, retrieve data regarding the heart failure risk level (e.g., patient metric data), modify the metric-specific thresholds used to determine the risk level, or conduct any other action related to the treatment of patient 14. In some examples, the clinician may be able to review raw data (e.g., higher resolution diagnostic information) to diagnose any other problems with patient 14 or monitor the efficacy of treatments given to patient 14. For example, the clinician may check if the intrathoracic impedance has increased after diuretic therapy or if the heart rate has decreased during atrial fibrillation in response to a rate controlling drug. User interface 104 may even suggest treatment along with the alert, e.g., certain drugs and doses, to minimize symptoms and tissue damage that could result from heart failure. User interface 104 may also allow the user to specify the type and timing of alerts based upon the severity or criticality of the heart failure risk level. In addition to the heart failure risk level, in other examples, user interface 104 may also provide the underlying patient metrics to allow the clinician to monitor therapy efficacy and remaining patient conditions.

In some examples, processor 100 of external device 24 and/or one or more processors of one or more networked computers may perform all or a portion of the techniques described herein with respect to processor 80, metric detection module 92 and IMD 16. For example, processor 100 or a metric detection module 92 within external device 24 may analyze patient metrics to detect those metrics exceeding thresholds and to generate the heart failure risk level.

FIG. 5 is a block diagram illustrating an example system that includes a server 114, such as a cloud based service, and one or more computing devices 120A-120N, that are coupled to the IMD 16 and external device 24 shown in FIG. 1 via a network 112. In the examples of the present disclosure described below, an external monitoring device, such as server 114, acquires and utilizes the diagnostic parameters, such as patient metrics, associated with diagnostic information from the remote implantable monitoring device, such as an IMD 16, and obtains and uses environmental factor information from a source such as environmental factor information source 125. Server 114 may use the patient metrics and environmental factor information to generate heart failure risk scores and differentiate between heart failure risk status alerts.

Server 114 may derive diagnostic metrics and/or diagnostic parameters using transmitted data from the implantable medical device 16. For example, Server 114 may process a transmitted electrocardiogram/electrogram (EKG/EGM) with an algorithm to derive diagnostic metrics and/or diagnostic parameters; either just from the current transmission or from a series of longitudinal transmissions to incorporate an understanding of deviation relative to each patient's baseline. Server 114 may apply a toolbox of detection/classification algorithms to EKG/EGM data to enrich the risk analysis relative to the IMD 16 metrics.

For example, once receipt of a scheduled data transmission from the remote implantable monitoring device is detected, the server may acquire patient metrics from the IMD that are used (along with the environmental factor information) by the server to determine an HFRS associated with the data transmission. An HFRS alert is then generated by the server based on the temporal proximity of a maximum daily HFRS within a lookback window prior to the current data transmission. For example, the external monitoring device may differentiate the alerts as being either high alerts, high ongoing alerts, medium alerts and medium ongoing alerts. In some examples, the server may differentiate HFRS as being high HFRS, a high new HFRS, a medium HFRS and a medium new HFRS.

Server 114 may obtain environmental factor information from environmental factor information source 125. Examples of environmental factors which have been shown to affect heart failure risk include air pollution, such as particulate matter concentration, and external temperature. Server 114 may send patient location information to the environmental factor information source 125 and receive the environmental information in response. In one example, server 114 uses an application programming interface (API) to access the environmental factor information at environmental factor information source 125. Server 114 may ensure that the location information is in a format accepted by the environmental factor information source 125, and may convert location in another format to format accepted by environmental factor information source 125 (such as by converting from coordinates to a zip code for example). Server 114 may do sanity checks on the environmental factor information received from environmental factor information source 125 such as obtaining the environmental information from multiple sources, checking the environmental information at other locations and times to ensure that the information from environmental factor information source 125 remains current, and clipping or ignoring environmental factor information that exceed a predetermined threshold.

Server 114 may obtain the location information from external device 24, such as a programmer or other device, or from patient computing device 129, such as a smart phone. Location information may be also obtained from a database that stores a patient's address provided by the patient or by a person or organization authorized by the patient.

Sensor 127 located near the patient may also be used to obtain environmental factor information and provide the environmental factor information to server 114. Sensor 127 may be a particulate matter sensor such as a laser-based sensor, optical sensor, or electrical mobility sensor. Sensor 127 may be an ozone sensor such as an electrochemical sensor, an ultraviolet (UV) photometer, or a spectrophotometer. Sensor 127 may be a temperature sensor such as a thermistor or an infrared (IR) sensor. FIG. 5 shows sensor 127 as being independent from other devices, but in some cases, sensor 127 may be part of another device, such as external device 24 or patient computing device 129.

In some examples, a HFRS notification, e.g., a new high, may be sent to a user according to their preferences, e.g., via email, pager, or text message (such as Short Message Service (SMS) or multimedia messages (MMS)). Moreover, processing circuitry of an IMD system may select a type of notification based on a determined HFRS differentiation, e.g., new versus ongoing, in some examples according to the techniques of this disclosure. The notification may indicate the HFRS, and in some cases the differentiation. For example, the processing circuitry may select a notification pushed to a personal device of a user (like patient computing device 129), e.g., a text message or email, for HFRS that are new and/or more severe, such as a new high HFRS, while other less new and/or severe HFRS notifications are collected and accessible at the user's convenience via a web service, e.g., the system of FIG. 5. The user may select a type of notification based on the HFRS differentiation, e.g., provide user preferences. For example, the user may only want to receive text messages for a new high and receive emails for all other alerts. In another example, the user may want to receive a message on their pager for all high new HFRS and medium new HFRS alerts and wait to access all other alerts via the web service.

Network 112 may be generally used to transmit diagnostic information (e.g., a risk level) from a remote IMD 16 to another external computing device so that a clinician or other healthcare professional may monitor patient 14. In this example, IMD 16 may use its telemetry module 88 to communicate with external device 24 via a first wireless connection, and to communication with an access point 110 via a second wireless connection. In the example of FIG. 5, access point 110, external device 24, server 114, and computing devices 120A-120N are interconnected, and able to communicate with each other, through network 112. In some cases, one or more of access point 110, external device 24, server 114, and computing devices 120A-120N may be coupled to network 112 through one or more wireless connections. IMD 16, external device 24, server 114, and computing devices 120A-120N may each comprise one or more processors, such as one or more microprocessors, DSPs, ASICs, FPGAs, programmable logic circuitry, or the like, that may perform various functions and operations, such as those described herein.

Access point 110 may comprise a device that connects to network 112 via any of a variety of connections, such as telephone dial-up, digital subscriber line (DSL), or cable modem connections. In other examples, access point 110 may be coupled to network 112 through different forms of connections, including wired or wireless connections. In some examples, access point 110 may be co-located with patient 14 and may comprise one or more programming units and/or computing devices (e.g., one or more monitoring units) that may perform various functions and operations described herein. For example, access point 110 may include a home-monitoring unit that is co-located with patient 14 and that may monitor the activity of IMD 16. In some examples, server 114 or computing devices 120 may control or perform any of the various functions or operations described herein, e.g., generate a heart failure risk level based on the patient metric comparisons or create patient metrics from the raw metric data.

Server 114 further includes input/output device 116, processor 118 and memory 119. Input/output device 116 includes input devices such as a keyboard, a mouse, voice input etc. and output device includes graphical user interfaces, output displays, printers and other suitable means. Processor 118 includes any suitable processor such as Intel Xeon Phi. Processor 118 is configured to set the start and end dates for each evaluation period. The evaluation period serves as an evaluation window that encompasses data, acquired from each patient, that are within the boundaries (i.e., start and end times). Processor 118 is also configured to perform a variety of calculations. For example, processor 118 calculates risk of HFH for each evaluation period. In one or more embodiments, weighting factors are applied to two or more evaluations periods to determine the risk.

Memory 119 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media. Exemplary data that may be stored in memory 119 includes heart failure patient data and heart failure prospective risk data. Evaluation period start and end times may also be stored in memory 119. Heart failure patient data includes data observations (e.g., data sensed from sensors that cross a threshold). Additionally, evaluation period data is also stored in memory 119. For example, the start and end dates of the evaluation period data is stored in memory 119.

In some cases, server 114 may be configured to provide a secure storage site for archival of diagnostic information (e.g., patient metric data and/or heart failure risk levels) that has been collected and generated from IMD 16 and/or external device 24. Network 112 may comprise a local area network, wide area network, or global network, such as the Internet. In some cases, external device 24 or server 114 may assemble the diagnostic information in web pages or other documents for viewing by and trained professionals, such as clinicians, via viewing terminals associated with computing devices 120. The system of FIG. 5 may be implemented, in some aspects, with general network technology and functionality similar to that provided by the Medtronic CareLink® Network developed by Medtronic, Inc., of Minneapolis, MN.

In the manner of FIG. 5, computing device 120A or external device 24, for example, may be remote computing devices that receive and present diagnostic information transmitted from IMDs of multiple patients so that a clinician may prioritize the patients needing treatment immediately. In other words, the clinician may triage patients by analyzing the heart failure risk levels of multiple patients. The computing device may use its communication module to receive the diagnostic information (e.g., heart failure risk levels) transmitted from multiple IMDs via network 112. In this manner, each heart failure risk level is representative of one the patients. Although the IMDs may transmit the diagnostic information at any time, generally the IMDs may transmit diagnostic information on a daily basis or in response to an interrogation request from an external computing device. In other examples, the IMDs may be configured to transmit diagnostic information when the risk level becomes critical or there is a medium or high risk of hospitalization within a predetermined period. A processor within the remote computing device may then automatically rank each of the patients based on each of the heart failure risk levels and the user interface may present the list of ranked patients to the clinician. Generally, the list will start with the most critical patient, e.g., the highest risk level, at the top. This method may be useful for healthcare professionals making house calls, serving patients within a nursing home, or any other circumstance in which a professional treats many patients. The healthcare professionals may thus triage patients in order to minimize any hospitalization due to heart failure.

In one example, a system, such as server 114, may comprise one or more input/output devices 116; and one or more processors 118 configured to receive a plurality of diagnostic parameters, such as patient metrics, of a patient associated with heart failure, wherein at least one of the plurality of diagnostic parameters is measured by and retrieved from an implantable medical device 16 and received from the implantable medical device. One or more processors 118 may be configured to obtain environmental factor information associated with heart failure for the patient, such as from environmental factor source 125 or from sensor 127. One or more processors 118 may determine a heart failure (HF) risk score indicating probability of occurrence of a heart failure event. The HF risk score may be derived using a model, such as a machine learning model like a Bayesian Belief Network, that uses the plurality of diagnostic parameters monitored over time and the environmental factor information; and in response to the HF risk score exceeding a threshold value, provide an alert or a suggestion to modify a therapy delivered to the patient by the implantable medical device 16 via the one or more input/output devices 116.

A Bayesian Belief Network (BBN) model may evaluate all the individual metric states for each patient metric and environmental factor information, combine the multiple patient metric states, and generate a probabilistic risk score for the heart failure risk level. The BBN approach allows for uncertain reasoning to approximate the likelihood of a heart rate failure hospitalization under a given set of patient metrics and environmental factor information. The score weight for each patient metric and environmental factor information may generate the HF risk score for the day using a lookup table defined by the BBN model using data from a development set. Also, the BBN model assumes that, in the absence of any information regarding the heart failure status, the patient metrics and environmental factor information are independent of each other.

The environmental factor information may be an air pollution indication such as particulate matter exposure level. The one or more processors may be configured to obtain the particulate matter exposure level from location information for the patient. The one or more processors may be configured to receive the location information for the patient, i.e., from personal computing device 129.

The environmental factor information may also be environmental temperature. The risk of a heart attack increases during periods of extreme cold or heat. Server 114 may obtain environmental temperature data for the patient from sensor 127 or from environmental factor information source 125. Locations and the swing in temperature exceeding a threshold potentially may also trigger HF worsening. Server 114 may track or derive such information from sensor 127 or from environmental factor information source 125.

Additional environmental factors may include allergens, such as pollens of different kinds. Allergens may be affected by patient susceptibility, some patients are sensitive to grass, some patients are sensitive to trees some to ragweed etc. Susceptible patients may be at a higher risk for HF worsening. Server 114 may keep track of patient allergen susceptibility and obtain allergen levels for such patients from sensor 127 or from environmental factor information source 125.

Further environmental factors may include localized factors that the patient has some control over. For example, sensor 127 can detect local environmental factors such as cigarette smoke, gas stoves emitting NOx, or other local environmental factors. Server 114 may use these local environmental factors in a determination of a heart failure (HF) risk score.

In addition to the level of an environmental factor, a change in an environmental factor may lead to a higher risk for HF worsening. Server 114 may process environmental factor information obtained for the patient from sensor 127 or from environmental factor information source 125 to produce such relevant environmental factor change data such as a day to day change in temperature or particulate matter.

The diagnostic parameters may include, as examples, therapy use statistics (e.g., pacing or shocks), thoracic impedance, heart rate, heart rate variability, patient activity, and a percentage of time receiving cardiac resynchronization therapy. Other example patient metrics include weight, blood pressure, respiration rate, sleep apnea burden (which may be derived from respiration rate), temperature, ischemia burden, the occurrence, frequency or duration cardiac events, and sensed cardiac intervals (e.g., heart rate or Q-T intervals). Examples of cardiac events may include atrial and ventricular tachyarrhythmias. Another example diagnostic parameter is the atrial tachycardia/fibrillation (AT/AF) burden and ventricular rate during AT/AF. The concentration or levels of various substances, such as blood glucose, hematocrit, troponin and/or brain natriuretic peptide (BNP) levels, within the patient may also be used as one or more diagnostic parameters.

The suggestion to modify therapy may comprise a suggestion to modify at least one of a substance delivered by an implantable pump, cardiac resynchronization therapy, refractory period stimulation, or cardiac potentiation therapy. Server 114 may provide such suggestions to clinicians along with alert and functionality at the server 114 enables the clinician to modify therapy provided by IMD 16 as need.

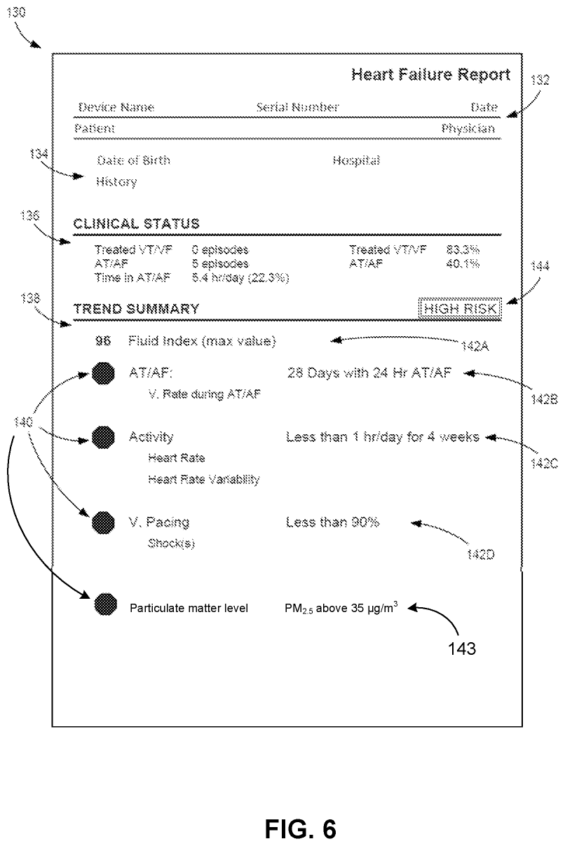

FIG. 6 illustrates an example screen 130 of user interface 104 that includes lower resolution diagnostic information. As shown in FIG. 6, screen 130 includes risk level 144 that indicates the risk that patient 14 will be hospitalized due to heart failure. Risk level 144 is an example of a periodic HFRS. As described herein, the heart failure risk level may be indicative that patient 14 would be hospitalized. Although screen 130 is described as being presented on user interface 104 of external device 24, screen 130 may be presented on any user interface of any device used by a healthcare professional. The heart failure report of screen 130 may be transmitted to a user at a scheduled frequency, e.g., once a day or once a week, or in response to an interrogation request from the user. As shown in FIG. 6, screen 130 is a heart failure report that includes identification data 132 and patient history data 134. Identification data 132 includes items such as the patient name, the device name, the serial number of IMD 16, the date, and even the physician name. Patient history data 134 may be relevant data that may help in the treatment of patient 14.

Screen 130 also includes clinical status 136 that includes information regarding the stimulation therapy delivered by IMD 16. Screen 130 also presents trend summary 138. Trend summary 138 presents a snapshot of certain patient metrics that are exceeding their respective metric thresholds to contribute to the severity of heart failure risk level 144. Critical indicator 140 is provided to remind the user that each of the patient metrics with critical indicator 140 is contributing to the heart failure risk level because the metric threshold has been met or exceeded. In examples in which risk level 144 is determined with a statistical analysis, critical indicator 140 may not be necessary. However, certain patient metrics that contribute significantly to the probability that patient 14 may be hospitalized may still be presented to the user.

In the example of FIG. 6, trend summary 138 presents four patient metrics 142A, 142B, 142C, and 142D (collectively “patient metrics 142”). Thoracic fluid index metric 142A indicates a maximum detected value of 96. Although thoracic fluid index metric 142A is not contributing to risk level 144 in this example, it is provided because it is an important indicator of thoracic fluid volume and potential heart failure. Atrial fibrillation duration 142B indicates that patient 14 has had 28 days of atrial fibrillation or atrial tachycardia for 24 hours. Activity metric 142C indicates that patient 14 has been active for less than 1 hour per day for the last 4 weeks. In addition, ventricular pacing metric 142D (e.g., a cardiac resynchronization therapy percentage) indicates that IMD 16 has been pacing heart 12 less than 90 percent of the time. As patient metrics 142 indicate, information may be given that is more specific than just a threshold has been exceeded. The actual observed patient metric data, or summary of the data, may be presented in trend summary 138.

Trend summary 138 may also include environmental factor 143. In the example of FIG. 6, the particulate matter level is above a threshold. In this case, PM_{2.5} is above 35 μg/m³. PM_{2.5} refers to fine particulate matter that is 2.5 micrometers in diameter or smaller. PM_{2.5} is of particular concern because it is small enough to inhale and can penetrate deep into the respiratory system and is, thus, often regulated and monitored by governmental agencies.

Since each of patient metrics 142B-D has exceeded their respective metric-specific threshold, critical indicator 140 is provided for each metric. Critical indicator 140 is also shown for environmental factor 143 which exceeds an environmental factor specific threshold. The user then knows that heart failure risk level 144 is generated with these critical patient metrics and environmental factor 143. Also, risk level 144 indicates that patient 14 is at “high risk” for being admitted to the hospital for heart failure. As described herein, risk level 144 may have two or more levels that indicate the severity of heart failure for patient 14. In some examples, “low risk” may indicate that no patient metrics have exceeded their respective metric-specific threshold, “medium risk” may indicate that one patient metric has exceeded their respective metric-specific thresholds, and “high risk” may indicate that two or more patient metrics have exceeded their respective metric-specific thresholds. Since three patient metrics, e.g., patient metrics 142B-D, have exceeded their respective metric-specific thresholds, risk level 144 is indicated as “high risk” in this example.

Risk level 144 is highlighted by a double-lined rectangle for easy location by the user. In other examples, risk level 144 may stand out from the rest of screen 130 in different manners. For example, risk level 144 may be of a different color, font size, or be presented with animation (e.g., flashing or scrolling). Alternatively, risk level 144 may be located at the top of screen 130 or other easily identifiable location. Although heart failure risk level 144 is generally presented as a word category, risk level 144 may be presented with a fraction, percentage, weighted average, or other numerical score that indicates that the severity of the heart failure risk level. For example, risk level 144 may be provided as a fraction of the critical patient metrics over the total number of observed patient metrics to give the user an immediate indication of the severity of the heart failure.

Although screen 130 may be a passively presented informational screen, screen 130 may be interactive. The user may select areas of screen 130 to view more details about any of patient metrics 142, e.g., the user may request higher resolution diagnostic information from IMD 16. Screen 130, in other examples, may provide scroll bars, menus, and navigation buttons to allow the user to view additional information, adjust therapy, adjust metric parameters, or perform other operations related to the treatment of patient 14 with the patient metrics and risk level.

In other examples, risk level 144 may be transmitted in a different manner. In other words, the diagnostic information may be transmitted without additional extraneous data such as patient metrics 142. For example, risk level 144 may be transmitted as a single data point associated with the name of patient 14. Alternatively, risk level 144 may be transmitted to a remote computing device as a data point and the remote computing device may update the risk level for that particular patient. In other examples, risk level 144 may be transmitted as part of a text message, electronic mail message, or other formatted message to a mobile computing device carried by a clinician or healthcare professional. After the user receives the diagnostic information, the user may send an interrogation request to IMD 16 for additional information, e.g., higher resolution diagnostic information.

Additional details of a system that uses diagnostic parameters from an IMD to determine a heart failure risk score is described in U.S. Patent Publication No. US2021/0204885 to Sharma et al., entitled, “DIFFERENTIATION OF HEART FAILURE RISK SCORES FOR HEART FAILURE MONITORING,” which published on Jul. 8, 2021 and is incorporated herein by reference in its entirety.

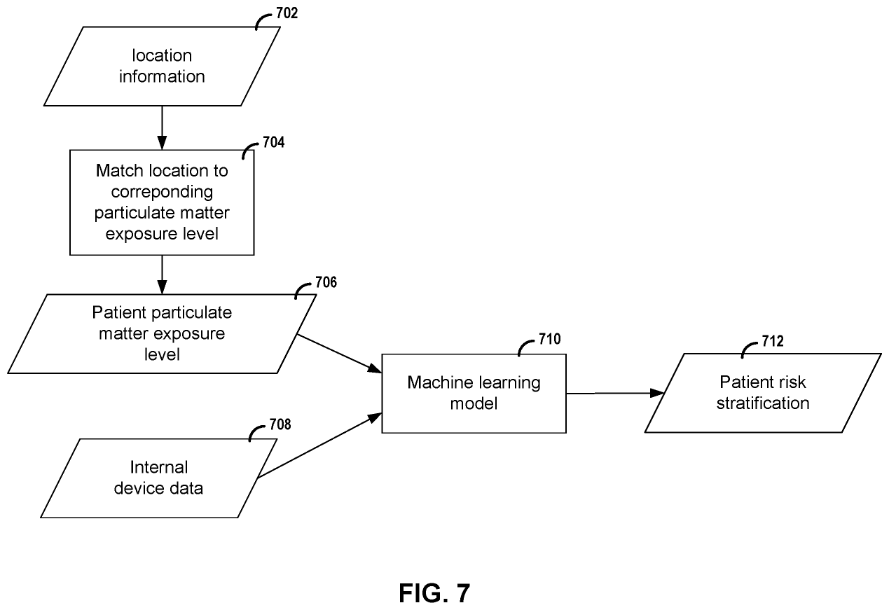

FIG. 7 illustrates an example method for patient risk stratification using internal device data and at least one environmental factor. The example of FIG. 7 shows particulate matter exposure level as the environmental factor but other relevant environmental factors may be used. In addition to environmental factors, the machine learning model 710 may be trained on information derived from the environmental factors, such as a change in particulate level or a change in temperature. Some of the environmental factors may be only trained on and only used for susceptible patients. For example, machine learning model 710 may be trained on allergen levels for susceptible patients and such an environmental factor only used for susceptible patients going forward. The example method of FIG. 7 may be implemented by a computing device such as server 114 shown in FIG. 5.

Patient location information 702 may be obtained from a patient device or a database as discussed above with respect to FIG. 5. Patient location information 702 may be any type of location data sufficient to obtain accurate particulate matter exposure level such as zip code, address, or latitude and longitude coordinates. In the example of FIG. 5 discussed above, server 114 may obtain the location information from external device 24, patient computing device 129 or a database.

In operation 704, the location information may be matched to a corresponding particulate matter exposure level 706. In the example of FIG. 5, server 114 may obtain environmental factor information from environmental factor information source 125. Server 114 may use an application programming interface (API) to access the environmental factor information at environmental factor information source 125. Environmental factor information source 125 may be a public source of particulate matter levels, such as AirNow.gov or waqi.info.

Machine learning model 710 may use particulate matter exposure level 706 as well as internal device data 708, such as patient metrics from an IMD, to create a patient risk stratification. Machine learning model 710 may be a Bayesian Belief Network which is a type of probabilistic model that represents the relationships between different variables and the probability of their occurrences. Bayesian Belief Networks may be used to model and analyze complex systems and help make predictions about the likelihood of different events occurring. In the context of risk assessment, a Bayesian Belief Networks may be used to model the relationships between different risk factors and the likelihood of a particular risk event occurring. Alternatives to Bayesian networks include other machine learning models, such as decision trees, random forests, support vector machines and neural networks, such as deep neural networks.

Machine learning model 710 may be trained using historical records of particulate matter exposure level as well as internal device data, e.g., for numerous subjects other than patient 14. In some examples, machine learning model 710 is trained with a training data set comprising numerous instances of particulate matter exposure level and internal device data labeled with heart failure risk levels. In this way, the relative importance of different internal device data sources such as patient metrics may be learned by machine learning model 710. Machine learning model 710 may be a machine learning model with parameters trained on patient metrics alone that is updated or tuned using the environmental factor source(s) information.

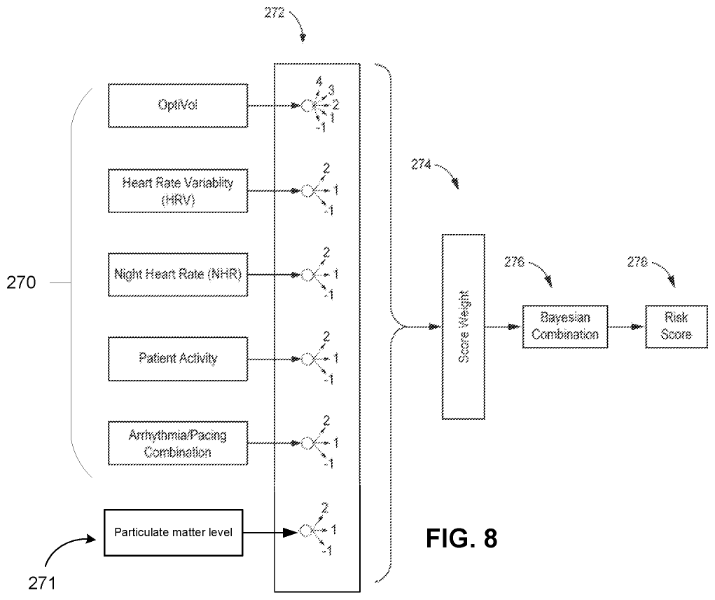

FIG. 8 is a flow diagram of example techniques for generating a heart failure risk score 278 using a Bayesian Belief Network mode 276. Heart failure risk score 278 is an example of a periodic HFRS. As shown in FIG. 8, each of the five patient metrics 270 and environmental factor 271 is detected and analyzed using criteria 272. Criteria 272 may be patient metric-specific thresholds. Based on criteria 272, score weights 274 for each patient metric is generated. Subsequently, score weights 274 are used to estimate the heart failure risk score 278 using the BBN combination model 276. Heart failure risk score 278 is categorized into three groups for analysis purposes: high, medium, and low. The “High” category was chosen to represent diagnostic evaluations associated with a greater risk that the patient will be hospitalized (e.g., a risk score of greater than 10% or 15%). The “Low” category was chosen to represent diagnostic evaluations where the metric state for all the patient metrics was mostly “Low” (e.g., a risk score of less than ˜5%). The “Medium” category comprises of all other metric state combinations that did not get classified as either “High” or “Low.”

Evaluations may be simulated, and each monthly evaluation may include: (1) looking back at the maximum value of the risk score in the last 30 days to determine the patient status into the diagnostic evaluation groups and (2) a prospective assessment for the first heart failure hospitalization in the next 30 days. The threshold for a high risk score was determined to be the first natural break after the top 10% of the risk score in the development set. Medium and low risk scores may be divided into two groups at a natural breakpoint with the heart failure hospitalization event rate <0.5% in the low group of the development set.

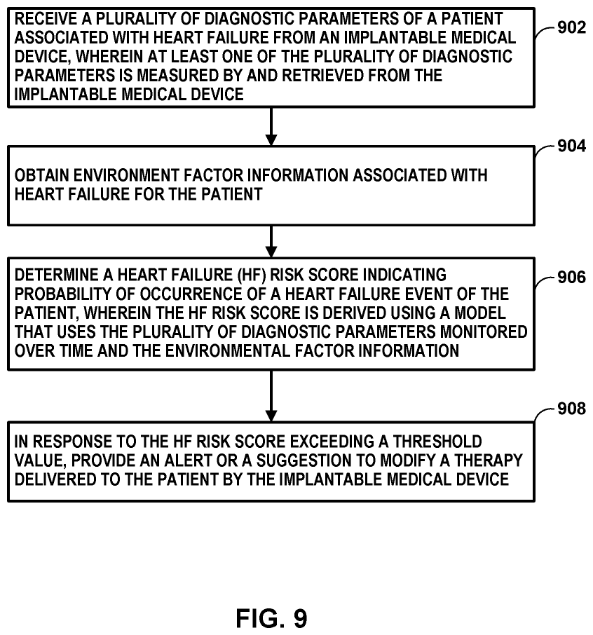

FIG. 9 is a flowchart that illustrates an example method for patient risk stratification using internal device data and at least one environmental factor. For purposes of illustration only, the operations of FIG. 8 are described below within the context of server 114 of FIG. 5.

Server 114 may receive a plurality of diagnostic parameters of a patient associated with heart failure from an implantable medical device 16, wherein at least one of the plurality of diagnostic parameters is measured by and retrieved from the implantable medical device 16 (902). Server 114 may obtain environmental factor information associated with heart failure for the patient (904). Server 114 may determine a heart failure (HF) risk score indicating probability of occurrence of a heart failure event of the patient, wherein the HF risk score is derived using a model that uses the plurality of diagnostic parameters monitored over time and the environmental factor information (906). Server 114 may, in response to the HF risk score exceeding a threshold value, provide an alert or a suggestion to modify a therapy delivered to the patient by the implantable medical device (908).

The techniques described herein describe techniques for enabling the high and medium alerts generated by the external monitoring device to be further differentiated as being either alerts or ongoing alerts, and, in some examples, by enabling further differentiation of an identified current HFRS alert as high, high new, medium and medium new. Such additional HFRS differentiation of the present disclosure may assist in prioritization of patients, determining patient conditions, assessing the effectiveness of a current therapy, and determining whether therapy adjustments may be necessary. For example, during monitoring of patients, knowing whether an event is an ongoing event, not an ongoing event, a new event, or not a new event enables more efficient prioritization and treatment of patients by the clinician.

Various examples have been described that include detecting and storing patient metrics and transmitting high and lower resolution diagnostic data from an IMD. These examples include techniques for identifying patients with an elevated risk of being hospitalized due to heart failure. In addition, an alert of patient risk levels may be remotely delivered to a healthcare professional from multiple different patients for triage and earlier diagnosis and treatment of heart failure before hospitalization. Any combination of detection and notification of heart failure risk level is contemplated. These and other examples are within the scope of the following claims.

1. A system comprising:
one or more input/output devices; and
one or more processors configured to:
receive, from an implantable medical device via the one or more input/output devices, a plurality of diagnostic parameters of a patient associated with heart failure, wherein at least one of the plurality of diagnostic parameters is measured by the implantable medical device;
obtain environmental factor information associated with heart failure for the patient via the one or more input/output devices;
determine a heart failure (HF) risk score indicating probability of occurrence of a heart failure event of the patient, wherein the HF risk score is derived using a model that uses the plurality of diagnostic parameters monitored over time and the environmental factor information; and
in response to the HF risk score exceeding a threshold value, provide an alert or a suggestion to modify a therapy delivered to the patient by the implantable medical device via the one or more input/output devices.

2. The system of claim 1, wherein the model is a Bayesian belief network.

3. The system of claim 1, wherein the environmental factor information comprises particulate matter exposure level.

4. The system of claim 1, wherein the one or more processors are configured to obtain the environmental factor information based on location information for the patient.

5. The system of claim 4, wherein the one or more processors are configured to receive the location information for the patient from a computing device of the patient.

6. The system of claim 1, wherein the environmental factor information comprises environmental temperature.

7. The system of claim 1, wherein the at least one of the diagnostic parameters comprises intrathoracic impedance.

8. The system of claim 1, wherein the at least one of the diagnostic parameters is derived at a server using transmitted data from the implantable medical device.

9. The system of claim 1, wherein the therapy comprises at least one of a substance delivered by an implantable pump, cardiac resynchronization therapy, refractory period stimulation, or cardiac potentiation therapy.

10. A method for determining a heart failure (HF) risk score of a patient indicating a probability of occurrence of a heart failure event, the method comprising:
receiving a plurality of diagnostic parameters of a patient associated with heart failure from an implantable device, wherein at least one of the plurality of diagnostic parameters is measured by and retrieved from the implantable medical device;
obtaining environmental factor information associated with heart failure for the patient;
determining a heart failure (HF) risk score indicating probability of occurrence of a heart failure event, wherein the HF risk score is derived using a model that uses the plurality of diagnostic parameters monitored over time and the environmental factor information; and
in response to the HF risk score exceeding a threshold value, providing an alert or a suggestion to modify a therapy delivered to the patient by the implantable medical device.

11. The method of claim 10, wherein the model is a Bayesian belief network.

12. The method of claim 10, wherein the environmental factor information comprises particulate matter exposure level.

13. The method of claim 10, further comprising obtaining the environmental factor information based on location information for the patient.

14. The system of claim 13, further comprising receiving the location information for the patient from a computing device of the patient.

15. The method of claim 10, wherein the environmental factor information comprises environmental temperature.

16. The method of claim 10, wherein the at least one of the diagnostic parameters comprises intrathoracic impedance.

17. The method of claim 10, wherein the at least one of the diagnostic parameters is derived at a server using transmitted data from the implantable medical device.

18. The method of claim 10, wherein the therapy comprises at least one of a substance delivered by an implantable pump, cardiac resynchronization therapy, refractory period stimulation, or cardiac potentiation therapy.

19. A system comprising:
an implantable medical device comprising one or more sensors configured to monitor over time at least one primary diagnostic parameter associated with heart failure; and
an external device interacting with the implantable medical device to obtain the at least one primary diagnostic parameter associated with heart failure; and
a server comprising:
a processor configured to:
receive the at least one primary diagnostic parameter from the implantable medical device;
obtain environmental factor information associated with heart failure for the patient;
determine a heart failure (HF) risk score indicating probability of occurrence of a heart failure event of the patient, wherein the HF risk score is derived using a model that uses the at least one primary diagnostic parameter monitored over time and the environmental factor information; and
in response to the HF risk score exceeding a threshold value, provide an alert or a suggestion to modify a therapy delivered to the patient by the implantable medical device.

20. The system of claim 19, wherein server comprises a cloud based server.
